(19) United States
(12) Patent Application Publication
(10) Pub. No.: US 2025/0152568 A1
(43) Pub. Date: May 15, 2025

(54) METHODS OF TREATING COMPLEX REGIONAL PAIN SYNDROME

(71) Applicant: Aisa Pharma, Inc., Boston, MA (US)

(72) Inventor: Andrew Sternlicht, Boston, MA (US)

(21) Appl. No.: 18/836,243

(22) PCT Filed: Feb. 6, 2023
(86) PCT No.: PCT/US2023/012388

§ 371 Date: Aug. 6, 2024

Related U.S. Application Data
(60) Provisional application No. 63/307,361, filed on Feb. 7, 2022.

(51) Int. Cl. A61K 31/4422; A61K 45/06

(52) U.S. Cl. CPC A61K 31/4422; A61K 45/06

(57) ABSTRACT

This disclosure relates to methods of treating, e.g., complex regional pain syndrome (CRPS) using dual N-type and L-type calcium channel blockers selective for the N-type calcium channel (e.g., cilnidipine).

### CROSS-REFERENCE TO RELATED APPLICATION

This application claims priority to U.S. Provisional Application No. 63/307,361, filed on Feb. 7, 2022, which is incorporated by reference herein in its entirety.

### TECHNICAL FIELD

This disclosure relates to methods of treating, e.g., complex regional pain syndrome (CRPS) using dual N-type and L-type calcium channel blockers selective for the N-type calcium channel (e.g., cilnidipine).

### BACKGROUND

Complex regional pain syndrome (CRPS) is a condition frequently characterized by pain and inflammation following an event such as injury to or surgery on a body part, such as an extremity. CRPS is classified as either Type I (previously referred to as reflex sympathetic dystrophy), which is about seven to eight times more prevalent than Type II, previously called ‘Causalgia’. In CRPS Type I, the syndrome occurs after an illness or injury that did not directly injure the nerves, such as immobilization, whereas in Type II a distinct nerve injury, such as a crush, precedes the development of the syndrome. About 70% of CRPS type I is “warm-type” CRPS where the affected extremity is warmer than the other extremity. The remaining 30% of CRPS type I is “cold-type” where the extremity is cooler than a non-affected limb. In many cases, patients having CRPS develop complete disability and loss of use of the affected appendage that can last for several years, and, in some cases, decades. The mental burden of CRPS is profound, with depression, anxiety, post traumatic stress disorder, insomnia, and substance abuse all occurring with alarming frequency in patients with the disease. The combined incidence of CRPS in the United States is less than 200,000 patients annually. Few therapeutic options are available for the treatment of CRPS, with many patients ultimately either requiring or resorting to opioid medications for relief.

### SUMMARY

Described herein are methods and compositions that include the use of dual N-type and L-type calcium channel blockers selective for the N-type calcium channel that can also, in some embodiments, possess sodium channel blocker activity (e.g., Nav 1.7 sodium channel blocker activity) or transient receptor potential vanilloid-1 ion channel (TRP-v1) blocker activity for the treatment of complex regional pain syndrome in a subject.

It is understood that the underlying cause of CRPS is, e.g., damage or dysfunction to peripheral sensory neurons (e.g., peripheral C-fiber nerve fibers), causing excess sympathetic overactivity and pain (e.g., neuropathic pain), inflammation, endothelial dysfunction, vasoconstriction, and increased oxidative stress. The neuropathic pain can, for example, be a burning pain, a pins and needles pain, thermal allodynia, thermal hyperalgesia, or a sensation of squeezing of the affected area. Secondary effects can occur, such as “mirror pain” that occurs on the opposite part or region of the body that corresponds to the affected area.

Multiple pathophysiologic pathways are understood to play a role in CRPS in patients. Therapeutic approaches that have shown some efficacy address a single or in some cases multiple pathways. Without wishing to be bound by theory, pathways that are understood to contribute to CRPS include:
A non-specific upregulated inflammatory state with increased production of inflammatory cytokines including TNF-α, IL-1B. IL2, and IL-6, increases in cGRP, bradykinin and substance P. Some of these cytokines are seen to be elevated in the cerebrospinal fluid as well as in the plasma of patients. Increased inflammation is both generalized as well as neurogenic.
An increased immunologic response with increases in non-specific autoantibodies to sympathetic receptors.
Dysregulation of the autonomic system with increases in sympathetic receptors and circulating catecholamines, sympathetic hyperactivity, and autonomic dysregulation.
Increased central sensitization and neuroplasticity with increased substance P, bradykinin, and glutamate; increased expression of spinal NMDA receptors; and possible gabaminergic increases in activity.
Increased peripheral sensitization with upregulation in TNF-α, and TRP-V1 activation.
Increased oxidative stress is present in patients with CRPS with upregulation of superoxide dismutase and other markers of oxidative stress and this also leads to immune system activation.
Microvascular pathology resulting from ischemia reperfusion injury associated with the original nerve injury which has been shown in animal models.
Endothelial dysfunction in “cold type” CRPS Type I.

N-type calcium channels are localized, e.g., at the sympathetic pre-synaptic nerve terminals and play a role in the release of neurotransmitters such as gamma-aminobutyric acid (GABA), acetylcholine, dopamine, and norepinephrine. N-type calcium channels are known to regulate, e.g., neuronal excitability and the firing of action potentials in the neurons, which increases the transmission of neurotransmitters in nociceptive pathways. These neurotransmitters then bind to the receptors on the sensory neurons that cause a person to feel pain. The induction of neuropathic pain can, in certain cases, be a result of the redistribution and alteration of subunit compositions of sodium and calcium channels that can result in spontaneous firing at abnormal locations along the sensory pathway. This may result in unpleasant sensory perceptions including, for example, burning pain, a feeling of wetness, itching, electrical shock pain, pain produced from normal stimulation, either from movement or just touch (e.g., mechanical and/or tactile allodynia), and the sensation of pins and needles.

Neuropathic pain is notoriously difficult to treat, with only 40-60% of patients achieving a degree of relief after treatment. Existing drugs have the potential for serious side effects that are, without wishing to be bound by theory, believed to be at least in part the result of unselective (e.g., non-discriminate or low selectivity) calcium channel inhibition.

Based on these considerations, N-selective dual N- and L-type calcium channel inhibition can be useful to treat diseases and disorders that are associated with dysregulation of blood flow and sympathetic nervous system overactivity, including those featuring symptoms of neuropathic pain.

A beneficial effect of L-type calcium channel inhibition is the dilation of the arteries in smooth muscle, causing an increase in arterial diameter, referred to as vasodilation. However, L-type calcium channel inhibition induces a homeostatic reflex mechanism in which norepinephrine is produced. The norepinephrine induces vasoconstriction, thus partially offsetting the vasodilating effects of the L-type calcium channel inhibition. A useful complementary effect of N-type calcium channel inhibition is the decrease of norepinephrine release and sympathetic outflow pre-synaptically in the spinal cord at the level of the dorsal root ganglion, which can counteract the homeostasis mechanism triggered by blockade of the L-type calcium channel. Disclosed herein are dual N-type and L-type calcium channel blockers selective for the N-type calcium channel (e.g., from about 5-fold to 50-fold to about 100-fold selective) which can, for example, (1) reduce neuropathic pain, (2) induce vasodilation, and (3) counter the homeostatic vasoconstriction triggered by blockade of the L-type calcium channel. Dual N-type and L-type calcium channel blockers selective for the N-type calcium channel are therefore particularly effective at treating, e.g., Raynaud's disease.

Selective inhibition of the N-type calcium channel has been shown to result in reduced severity and/or frequency of side effects and increased tolerability compared to non-N-selective calcium channel blockade. Further, dual N-type and L-type calcium channel blockers selective for the N-type calcium channel may be effective for certain conditions at lower dosages and may provide higher efficacy relative to less selective calcium channel blockers at equipotent doses as regards their blood pressure lowering effects in hypertensive individuals. Additional advantages include an increase in bone density in certain subjects (e.g., subjects afflicted with osteoporosis) and beneficial renal effects. The beneficial renal effects are, without wishing to be bound by theory, believed to be an effect of reduced renal constriction, improvement in renal podocyte functioning, and improved blood flow in the kidney.

Additional advantages of selective N-channel blockade by dual N-type and L-type calcium channel blockers selective for the N-type calcium channel, compared to calcium channel blockers that lack N-channel selectivity, can include:
Improvement in endothelial function and endothelial concentrations of nitric oxide by improving blood flow, reducing pain that is, e.g., a consequence of reduced blood flow.
Improvement in cardiac and left ventricle functioning resulting in reduction of pain due, e.g., to ischemia.
Improvement in the incidence and severity of atherosclerosis including reducing pain caused, e.g., by a reduction in blood flow, and reducing the overall incidence of atherosclerotic-related events.
Decrease in overall sympathetic nervous system activity and plasma concentration of norepinephrine, which can decrease pain due to net arteriole dilation and decrease in sympathetically mediated pain syndromes.
Improvement in overall autonomic functioning, which may improve gut function in patients whose gut function (e.g., competency of the lower esophageal sphincter and peristalsis and gastric emptying), is compromised due to impaired autonomic function as occurs in certain disease states (e.g., scleroderma).
Decrease in oxidative stress and reduction in reactive oxygen species.
Improvement in organ injury after a period of relative or localized ischemia and subsequent reperfusion.

Dual N-type and L-type calcium channel blockers selective for the N-type calcium channel are also amenable to combination with other agents which may have an additive or complementary effect with the dual N-type and L-type calcium channel blockers selective for the N-type calcium channel. For example, dual N-type and L-type calcium channel blockers selective for the N-type calcium channel may reduce the blood pressure of a hypertensive subject, which can be counterbalanced by combining the dual N-type and L-type calcium channel blockers selective for the N-type calcium channel with an agent that increases blood pressure. Without wishing to be bound by theory, non N-channel specific dihydropyridine calcium channel antagonists as well as the dual N-type and L-type calcium channel blockers selective for the N-type calcium channel do not typically appreciably lower blood pressure in normotensive subjects (i.e., subjects that do not have abnormal blood pressure; e.g., a subject that does not have hypertension). Further, the vasodilating effects of dual N-type and L-type calcium channel blockers selective for the N-type calcium channel can improve the effectiveness of other vasodilators. Cilnidipine exerts a balance of selective N-vs. L-type calcium channel inhibition (which can have a 5 fold to 50-fold to 100-fold selectivity for N-type calcium channel over L-type calcium channel), making it surprisingly effective at treating diseases and disorders characterized by neuropathic pain and vasoconstriction.

Dual N-type and L-type calcium channel blockers selective for the N-type calcium channel, such as cilnidipine, are also understood to have activity at the Nav 1.7 sodium channel, and likely have activity at the TRP-v1 receptor (i.e., the capsaicin receptor) as has been shown with N-channel antagonists. Nav 1.7 channels are expressed in the nociceptive neurons at dorsal root ganglion, geminal ganglion, and sympathetic ganglion neurons, which are part of the autonomic nervous system and mediate pain, thus providing cilnidipine an additional mode of action for alleviating pain. It has been demonstrated that a 20 mg dose of cilnidipine provides >6-fold inhibition of Nav 1.7 compared to an 1800 mg dose of gabapentin. See, for example, published PCT application WO2021/178903 which is incorporated by reference herein in its entirety.

The TRP-v1 receptor is located at peripheral nociceptors and mediates conditions characterized by thermal hypersensitivity (e.g., thermal allodynia and thermal hyperalgesia). The inhibition of TRP-v1 would thus provide a useful complementary effect against thermal hypersensitivity symptoms seen in CRPS patients.

In some aspects, disclosed herein is a method of treating complex regional pain syndrome in a subject in need thereof, comprising administering to the subject a therapeutically effective amount of a dual N-type and L-type calcium channel blocker selective for the N-type calcium channel.

In some aspects, disclosed herein is a method of treating pain in a subject in need thereof, comprising administering to the subject a therapeutically effective amount of a dual N-type and L-type calcium channel blocker selective for the N-type calcium channel; wherein the subject has complex regional pain syndrome.

Other aspects of the invention will become apparent by consideration of, e.g., the detailed description and claims.

### Definitions

As used herein, the terms “about” and “approximately” are used interchangeably, and when used to refer to modify a numerical value, encompass a range of uncertainty of the numerical value of from 0% to 10% of the numerical value.

As used herein, the singular forms “a,” “an,” and “the” include plural referents unless the context clearly dictates otherwise.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the disease or disorder is associated with dysregulation of blood flow and sympathetic nervous system overactivity. In some embodiments, the disease or disorder is characterized by neuropathic pain, vasoconstriction, dysesthetic pain, burning pain, body temperature changes of the subject, hyperesthesia, changes in skin or tissue color, edema, changes in skin turgor, ruble, pallor, cyanosis, vasospasm, or any combination thereof.

As used herein, the phrase “fixed dosage form” refers to the simultaneous administration of a dual N-type and L-type calcium channel blocker selective for the N-type calcium channel and at least one additional therapeutic agent (e.g., a calcineurin inhibitor, a non-steroidal anti-inflammatory drug, or both to a subject in the form of a single composition or dosage.

As used herein, the term “dual N-type and L-type calcium channel blocker selective for the N-type calcium channel”, “selective N-type calcium channel blocker”, “selective N-type CCB”, and “N-type selective CCB” refer to an agent that inhibits both N- and L-type calcium channels, and inhibits the N-type calcium channel to a greater degree than the L-type calcium channel. In some embodiments, the dual N-type and L-type calcium channel blocker selective for the N-type calcium channel has at least a 5-fold selectivity for the N-type calcium channel over the L-type calcium channel. For example, the dual N-type and L-type calcium channel blocker selective for the N-type calcium channel exhibits at least a 10-fold, at least a 30-fold, at least a 50-fold, at least a 80-fold, at least a 100-fold, at least a 300-fold, at least a 500-fold, at least a 800-fold, at least a 900-fold, or at least a 1000-fold selectivity for the N-type calcium channel over the L-type calcium channel. For example, the dual N-type and L-type calcium channel blocker selective for the N-type calcium channel exhibits at least a 50-fold to 100-fold selectivity for the N-type calcium channel over the L-type calcium channel. In some embodiments, the dual N-type and L-type calcium channel blocker selective for the N-type calcium channel exhibits a greater selectivity for the N-type calcium channel over the L-type calcium channel than a non-N-selective calcium channel blocker. In some embodiments, the non-N-selective calcium channel blocker is amlodipine, nifedipine, nicardipine, nimodipine, diltiazem or verapamil. In some embodiments, the dual N-type and L-type calcium channel blocker selective for the N-type calcium channel exhibits at least a 5% (e.g., at least a 10%, 20%, 30%, 40%, 50%, 60%, 70%, 80%, 100%, 150%, 200%, 300%) greater selectivity for the N-type calcium channel over the L-type calcium channel than a non-N-selective calcium channel blocker. Examples of dual N-type and L-type calcium channel blocker selective for the N-type calcium channel include, but are not limited to, cilnidipine, Z-160, ralfinamide, CNV2197944, or pharmaceutically acceptable salts thereof. Examples of dual N-type and L-type calcium channel blocker selective for the N-type calcium channel include, but are not limited to, cilnidipine, Z-160, CNV2197944, or pharmaceutically acceptable salts thereof. Examples of dual N-type and L-type calcium channel blocker selective for the N-type calcium channel include, but are not limited to, cilnidipine, Z-160, zicinotide, and pharmaceutically acceptable salts thereof.

In some embodiments, the dual N-type and L-type calcium channel blocker selective for the N-type calcium channel is also a Nav 1.7 sodium channel blocker (i.e., the dual N-type and L-type calcium channel blocker selective for the N-type calcium channel further inhibits a Nav 1.7 sodium channel). In some embodiments, the dual N-type and L-type calcium channel blocker selective for the N-type calcium channel that further inhibits a sodium channel (e.g., Nav 1.7) is cilnidipine. In some embodiments, the dual N-type and L-type calcium channel blocker selective for the N-type calcium channel is also a TRP-v1 channel inhibitor (i.e., the dual N-type and L-type calcium channel blocker selective for the N-type calcium channel further inhibits a TRP-v1 channel). In some embodiments, the dual N-type and L-type calcium channel blocker selective for the N-type calcium channel that further inhibits a TRP-v1 channel inhibitor is cilnidipine.

As used herein, the terms “non-N-type selective calcium channel blocker” and “non-N-selective calcium channel blocker” refer to an agent that blocks one or more calcium channels, but either (1) does not block the N-type calcium channel, or (2) blocks the N-type calcium channel, but not selectively over the L-type calcium channel. Examples of non-N-type selective calcium channel blockers include, but are not limited to, nifedipine, nicardipine, amlodipine, Z-944, nimodipine, verapamil, diltiazem, felodipine, isradipine, nisoldipine, mibafredil, nilvidipine barnidipine, benidipine lacidipine, lercanidipine, manidipine and nitrendipine, and pharmaceutical salts thereof.

As used herein, the term “Nav 1.7 sodium channel blocker” or “Nav 1.7 sodium channel inhibitor” refers to an agent that can inhibit the Nav 1.7 sodium channel. In some embodiments, the Nav 1.7 sodium channel blocker inhibits the closed state of the Nav 1.7 sodium channel. In some embodiments, the Nav 1.7 sodium channel blocker inhibits the inactivated state of the Nav 1.7 sodium channel. In some embodiments, the Nav 1.7 sodium channel blocker is also a dual N-type and L-type calcium channel blocker selective for the N-type calcium channel (i.e., the Nav 1.7 sodium channel blocker further inhibits the N-type and L-type calcium channels selectively for the N-type calcium channel). In some embodiments, the Nav 1.7 sodium channel blocker is also a TRP-v1 inhibitor (i.e., the Nav 1.7 sodium channel blocker further inhibits a TRP-v1 channel). In some embodiments, the Nav 1.7 sodium channel blocker that further inhibits the N-type and L-type calcium channels selectively for the N-type calcium channel is cilnidipine.

As used herein, the term “TRP-v1 inhibitor” refers to an agent that can inhibit the TRP-v1 channel. In some embodiments, the TRP-v1 inhibitor is also a dual N-type and L-type calcium channel blocker selective for the N-type calcium channel (i.e., the TRP-v1 inhibitor further inhibits the N-type and L-type calcium channels selectively for the N-type calcium channel). In some embodiments, the TRP-v1 inhibitor is also a Nav 1.7 sodium channel blocker (i.e., the TRP-v1 inhibitor further inhibits the Nav 1.7 sodium channel).

As used herein, the term “adverse effect” refers to an undesirable effect resulting from an alteration in normal physiology in a subject.

As used herein, the term “vasoconstriction” refers to the reduction in diameter of a blood vessel (e.g., an artery, vein, or capillary) resulting in reduced blood flow to the tissue the vasoconstricted blood vessels circulate blood to and from.

As used herein, the term “body temperature” refers to the temperature range of the body and/or one or more parts of the body in a healthy, awake subject under normal conditions of thermoregulation as measured, for example, in the mouth, the rectum, the armpit, the hands, the feet, or the ear. For example, the temperature range in a healthy human subject under normal conditions of thermoregulation in the rectum, heart, oropharynx, tympanic membrane, and esophagus is 36.1° C. to 37.8° C.; and the temperature range in a healthy human subject under normal conditions of thermoregulation in the hand or foot (e.g., hand) can be 18° C. to 37.8° C., depending on the ambient temperature.

The term “therapeutically effective amount,” as used herein, refers to a sufficient amount of a chemical entity (e.g., a dual N-type and L-type calcium channel blocker selective for the N-type calcium channel) being administered which will relieve to an extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: transdermal, intranasally, sublingual, intraspinal, or ocular administration.

The term “abnormally” when used, for example, in the terms “abnormally high”, “abnormally elevated”, or “abnormally low”, means a deviation from the range of the parameter being referred to that is found in a healthy subject as would be recognized by a medical professional, and that can be considered as indicative or predictive of dysfunction or a pathological state. Further, “abnormal” can, in some embodiments, refer to a physiologic response that is persistent beyond when a normal person would have recovered from that response; or a physiologic response that is exaggerated in degree and/or duration relative to what occurs in a normal, healthy subject.

For purposes of clarification, unless otherwise specified herein, when a variable (e.g., condition, feature, state, parameter, score, or statistic) in a subject is increased, decreased, or improved, the increase, decrease, or improvement is, for example, measured, assessed, or obtained in relation to the same variable measured, assessed, or obtained before the start of treatment (e.g., before administration of the dual N-type and L-type calcium channel blocker selective for the N-type calcium channel), unless otherwise specified herein. The variable can be a single measurement, assessment, or score; an average of a plurality of measurements, assessments, or scores; or a daily average of a plurality of measurements, scores, or assessments. Unless otherwise specified herein, measurements, assessments, or scores are typically taken within 1 month (e.g., within 3 weeks, 2 weeks, 10 days, 9 days, 8 days, 7 days, 6 days, 5 days, 4 days, 3 days, 2 days, 1 day, 18 hours, 12 hours, or 6 hours) of the administration of the dual N-type and L-type calcium channel blocker selective for the N-type calcium channel and/or the phosphodiesterase type 5 inhibitor. For example, reducing the frequency of symptoms in a subject can occur, e.g., when the number or average number of symptomatic episodes perceived by the subject that occurred during a span of time after administration of the dual N-type and L-type calcium channel blocker selective for the N-type calcium channel is less than the number or average number of symptomatic episodes perceived by the subject that occurred during the same span of time before administration of the dual N-type and L-type calcium channel blocker selective for the N-type calcium channel.

The details of one or more embodiments of the invention are set forth in the description below. Other features and advantages of the invention will be apparent from the description and drawings, and from the claims.

### DETAILED DESCRIPTION

In some aspects, disclosed herein is a method of treating complex regional pain syndrome in a subject in need of treatment thereof, comprising administering to the subject a therapeutically effective amount of a dual N-type and L-type calcium channel blocker selective for the N-type calcium channel.

In some aspects, disclosed herein is a method of treating pain in a subject in need thereof, comprising administering to the subject a therapeutically effective amount of a dual N-type and L-type calcium channel blocker selective for the N-type calcium channel; wherein the subject has complex regional pain syndrome.

Examples of N-type calcium channels include, but are not limited to, the Cav2.2 Type, which has two subunits, Cav 2.2a and Cav2.2b, both of which have an alpha 1 subunit of 2.2 and are affected by N type current.

Without wishing to be found by theory, the complex regional pain syndrome can be identified or diagnosed in any region of the body. In some embodiments, the complex regional pain syndrome is identified or diagnosed in a hand, upper extremity, a foot, or a lower extremity (e.g., the right hand or the left hand) of the subject. In some embodiments, the complex regional pain syndrome is identified or diagnosed in a finger (e.g., pollicus, phalange 2, phalange 3, phalange 4, or phalange 5) of the subject. In some embodiments, the complex regional pain syndrome is identified or diagnosed in an arm (e.g., the right arm or the left arm) of the subject. In some embodiments, the complex regional pain syndrome is identified or diagnosed in the neck of the subject. In some embodiments, the complex regional pain syndrome is identified or diagnosed in the head (e.g., the face) of the subject. In some embodiments, the changes associated with complex regional pain syndrome can be identified in the brain of the subject. In some embodiments, changes associated with complex regional pain syndrome can be identified in the hearing of the subject, which can, in some embodiments, result in hyperacusis. In some embodiments, the complex regional pain syndrome is identified or diagnosed in the torso (e.g., the chest, the back, or the abdomen) of the subject. In some embodiments, the complex regional pain syndrome is identified or diagnosed in the buttocks of the subject. In some embodiments, the complex regional pain syndrome is identified or diagnosed in a leg (e.g., the right leg or the left leg) of the subject. In some embodiments, the complex regional pain syndrome is identified or diagnosed in a foot (e.g., the right foot or the left foot) of the subject. In some embodiments, the complex regional pain syndrome is identified or diagnosed in or underneath the skin of the subject. In some embodiments, the complex regional pain syndrome is identified or diagnosed in the muscle of the subject. In some embodiments the CRPS can occur in the breast or genital areas of the subject. In some embodiments, the complex regional pain syndrome is identified or diagnosed in an organ of the subject.

In some embodiments, the complex regional pain syndrome is associated with an injury, stroke, heart attack, inflammation, and/or nervous system dysfunction in the subject. In some embodiments, the injury, stroke, heart attack, inflammation, and/or nervous system dysfunction that the complex regional pain syndrome is associated with manifested in the subject (e.g., was identified or diagnosed in the subject) before (for example, from about 1 day to 1 week before, from about 1 week to about 2 weeks before, from about 2 weeks to about 3 weeks before, from about 3 weeks to about 4 weeks before, from about 1.5 to about 3.5 weeks before, from about 1.5 to about 2.5 weeks before, from about 2 to about 3 weeks before, about 1 day before, about 2 days before, about 3 days before, about 4 days before, about 5 days before, about 1 week before, about 1.5 weeks before, about 2 weeks before, about 2.5 weeks before, about 3 weeks before, or about 3.5 weeks before) manifestation of the complex regional pain syndrome. In some of these embodiments, the injury, stroke, heart attack, inflammation, and/or nervous system dysfunction that the complex regional pain syndrome is associated with is a cause of the complex regional pain syndrome. It is understood that the injury, stroke, heart attack, inflammation, and/or nervous system dysfunction in the subject may, for example, result in improper function of the peripheral C-fiber nerve fibers that carry pain messages to the brain, thus resulting in the complex regional pain syndrome. Their excess firing also triggers inflammation designed to promote healing and rest after injury. In some embodiments, the complex regional pain syndrome is associated with an injury in the subject. In some embodiments, the complex regional pain syndrome is associated with a bone fracture, a cut, a burn, a sprain, a surgery, or a crushing injury. In some embodiments, the complex regional pain syndrome is associated with a bone fracture. In some embodiments, the complex regional pain syndrome is associated with a cut. In some embodiments, the complex regional pain syndrome is associated with a burn. In some embodiments, the complex regional pain syndrome is associated with a sprain. In some embodiments, the complex regional pain syndrome is associated with a surgery. In some embodiments, the complex regional pain syndrome is associated with a crushing injury. In some embodiments, the complex regional pain syndrome is associated with a bone fracture or a crushing injury to a nerve. In some embodiments, the complex regional pain syndrome is associated with a crushing injury to a nerve. In some embodiments, the complex regional pain syndrome is associated with a stroke in the subject. In some embodiments, the complex regional pain syndrome is associated with a heart attack in the subject. In some embodiments, the complex regional pain syndrome is associated with inflammation in the subject. In some embodiments, the complex regional pain syndrome is associated with a nervous system dysfunction in the subject.

In some embodiments, the subject is a female. In some embodiments, the subject is a male. In some embodiments, the age of the subject is from 20 to 60 (e.g., from 25 to 55, from 25 to 50, from 25 to 45, from 25 to 40, from 25 to 35, from 35 to 60, from 35 to 55, from 35 to 50, from 35 to 45, from 37 to 43, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60).

In some embodiments, the subject was diagnosed or identified as having abnormal (e.g., above average) anxiety. In some embodiments, the subject was diagnosed or identified as having depression.

In some embodiments, the complex regional pain syndrome is complex regional pain syndrome Type I. In some embodiments, the complex regional pain syndrome is complex regional pain syndrome Type II. In some embodiments, a nerve (e.g., a median nerve, a tibial nerve, an ulnar nerve and/or a sciatic nerve) in the subject is damaged. In some embodiments, a nerve is not damaged.

In some embodiments, after administration of the therapeutically effective amount of the dual N-type and L-type calcium channel blocker selective for the N-type calcium channel, the severity, frequency, and/or duration of pain (e.g., burning pain), sensitivity to touch or cold, swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, bone weakening, joint damage or abnormalities, cold skin, red skin, lack of sensation, edema, vasoconstriction, or poor motor skills or any combination thereof are reduced in the subject. In some embodiments, after administration of the therapeutically effective amount of the dual N-type and L-type calcium channel blocker selective for the N-type calcium channel, the severity, frequency, and/or duration of pain (e.g., burning pain), swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, bone weakening, joint damage or abnormalities, cold skin, red skin, lack of sensation, edema, vasoconstriction, or poor motor skills or any combination thereof are reduced in the subject. In some embodiments, changes in skin temperature, changes in skin texture, changes in skin color, changes in growth rate of hair and/or nails are understood to be abnormal changes as deemed by a medical professional. In some embodiments, after administration of the therapeutically effective amount of the dual N-type and L-type calcium channel blocker selective for the N-type calcium channel, the severity, frequency, and/or duration of pain (e.g., burning pain), sensitivity to cold, changes in skin temperature, changes in skin texture, changes in skin color, changes in growth rate of hair and/or nails, or any combination thereof are reduced in the subject. In some embodiments, after administration of the therapeutically effective amount of the dual N-type and L-type calcium channel blocker selective for the N-type calcium channel, the severity, frequency, and/or duration of pain (e.g., burning pain) is reduced in the subject. In some embodiments, after administration of the therapeutically effective amount of the dual N-type and L-type calcium channel blocker selective for the N-type calcium channel, the severity, frequency, and/or duration of sensitivity to cold is reduced in the subject. In some embodiments, after administration of the therapeutically effective amount of the dual N-type and L-type calcium channel blocker selective for the N-type calcium channel, the severity, frequency, and/or duration of changes in skin temperature are reduced in the subject. In some embodiments, after administration of the therapeutically effective amount of the dual N-type and L-type calcium channel blocker selective for the N-type calcium channel, the severity, frequency, and/or duration of changes in skin texture are reduced in the subject. In some embodiments, after administration of the therapeutically effective amount of the dual N-type and L-type calcium channel blocker selective for the N-type calcium channel, the severity, frequency, and/or duration of changes in skin color are reduced in the subject. In some embodiments, after administration of the therapeutically effective amount of the dual N-type and L-type calcium channel blocker selective for the N-type calcium channel, the severity, frequency, and/or duration of changes in growth rate of hair and/or nails are reduced in the subject. In some embodiments, after administration of the therapeutically effective amount of the dual N-type and L-type calcium channel blocker selective for the N-type calcium channel, the severity, frequency, and/or duration of burning or throbbing pain, sensitivity to cold, swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, or any combination thereof are reduced in the subject. In some embodiments, the pain is neuropathic pain. In some embodiments, the pain is selected from burning pain, hyperpathia, hyperalgesia, and allodynia (e.g., thermal allodynia). In some embodiments, the pain is selected from burning pain, hyperpathia, and thermal allodynia. In some embodiments, the pain is burning pain. In some embodiments, the pain is chronic pain. In some embodiments, the pain intensifies upon exposure of the subject to stress, sound, or light; or when the subject exercises.

In some embodiments, the subject does not have mechanical hyperesthesia, mechanical allodynia, mechanical hypersensitivity, or sensitivity to touch. In some embodiments, the subject does not have hyperesthesia, allodynia, or hypersensitivity. In some embodiments, the subject does not have neuropathic pain.

In some embodiments, the treating comprises alleviating one or more symptoms associated with complex regional pain syndrome in the subject. In this context, alleviating one or more symptoms associated with complex regional pain syndrome can, for example, comprise reducing the severity, duration, and/or frequency of the symptoms when compared to (1) the severity, duration, and/or frequency of the one or more symptoms in the subject before start of the treatment (e.g., before administration of the dual N-type and L-type calcium channel blocker selective for the N-type calcium channel, and wherein the severity, duration, and/or frequency of the one or more symptoms before administration of the dual N-type and L-type calcium channel blocker selective for the N-type calcium channel can, for example, be evaluated by a single measurement or assessment, or an average of a plurality of measurements or assessments taken, e.g., over the course of a 2 week period, a 7 day period, a 6 day period, a 5 day period, a 4 day period, a 3 day period, a 2 day period, or a 1 day period (e.g., a 7 day period)), wherein, for example, the reduction in severity, duration, and/or frequency of the symptoms is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 6 weeks, 2 months, 3 months, or 1 year of treatment); (2) the severity, duration, and/or frequency of the one or more symptoms experienced by a subject after the subject was administered a placebo; and/or (3) the severity, duration, and/or frequency of the one or more symptoms experienced by a subject after the subject was administered an alternative treatment such as a non-N selective calcium channel blocker. In some embodiments, the reduction in severity, duration, and/or frequency of the symptoms is greatest within 2 days (e.g., within 1.5 days, within 1 day, within 20 hours, within 16 hours, within 12 hours, within 8 hours, within 6 hours, within 4 hours, within 2 hours, or within 1 hour after administration of the dual N-type and L-type calcium channel blocker selective for the N-type calcium channel. In some embodiments, the reduction in severity, duration, and/or frequency of the symptoms is greatest within 8 hours after administration of the dual N-type and L-type calcium channel blocker selective for the N-type calcium channel.

In some embodiments, after administration of the dual N-type and L-type calcium channel blocker selective for the N-type calcium channel in the subject, intracellular calcium influx in tissue and/or macrophages is reduced. It is understood that the reduction in intracellular calcium levels is a direct result of blockade of calcium channels and furthermore can be attributed to inhibition of the TRPV-1 peripheral nociceptor ending.

In some embodiments, the non-N-selective calcium channel blocker is a dihydropyridine. In some other embodiments, the non-N-selective calcium channel blocker is a non-dihydropyridine. Non-limiting examples of non-N-selective calcium channel blockers include, but are not limited to: nifedipine, nicardipine, amlodipine, Z-944, nimodipine, verapamil, diltiazem, felodipine, isradipine, nisoldipine, mibafredil, nilvidipine barnidipine, benidipine lacidipine, lercanidipine, manidipine and nitrendipine, and pharmaceutically acceptable salts thereof. In some embodiments, the non-N-selective calcium channel blocker is amlodipine, nifedipine, nicardipine, nimodipine, diltiazem or verapamil.

In some aspects, disclosed herein is a method of reducing the frequency of one or more symptoms associated with complex regional pain syndrome in a subject, comprising administering to the subject a therapeutically effective amount of a dual N-type and L-type calcium channel blocker selective for the N-type calcium channel.

In some aspects, disclosed herein is a method of reducing the severity of one or more symptoms associated with complex regional pain syndrome in a subject, comprising administering to the subject a therapeutically effective amount of a dual N-type and L-type calcium channel blocker selective for the N-type calcium channel.

In some aspects, disclosed herein is a method of reducing the duration of one or more symptoms associated with complex regional pain syndrome in a subject, comprising administering to the subject a therapeutically effective amount of a dual N-type and L-type calcium channel blocker selective for the N-type calcium channel.

In some embodiments, symptoms associated with complex regional pain syndrome include, but are not limited to, pain, muscle atrophy (e.g., in the appendages), bone weakening, joint damage, cold skin, red skin, lack of sensation, edema, vasoconstriction, sudomotor abnormalities (e.g., excessive perspiration), or poor motor skills. In some embodiments, the pain is neuropathic pain. In some embodiments, the pain is selected from burning pain, hyperpathia, hyperalgesia, and allodynia. In some embodiments, the pain is selected from burning pain, hyperpathia, thermal hyperalgesia, and thermal allodynia. In some embodiments, the pain is burning pain. In some embodiments, the pain is chronic pain. In some embodiments, the pain intensifies upon exposure of the subject to stress, sound, or light; or when the subject exercises.

In some embodiments, after administration of the dual N-type and L-type calcium channel blocker selective for the N-type calcium channel autonomic function of the subject is improved; wherein the improvement of autonomic functioning in the subject is characterized by a lower reduction in systolic blood pressure in the upper arm (e.g., the portion of the arm between the elbow and shoulder, inclusive of the elbow and shoulder) of the subject when the subject is subjected to a tilt table test. For information on the tilt table test, see, for example, Clin. Auton. Res. 2019, 29 (2), 215-230, which is incorporated herein in its entirety.

In some embodiments, after administration of the dual N-type and L-type calcium channel blocker selective for the N-type calcium channel in the subject, one or both of sympathetic tone diminution and direct smooth muscle relaxation, occur in the subject.

In some embodiments, vasoconstriction in the subject is reduced after administering the dual N-type and L-type calcium channel blocker selective for the N-type calcium channel to the subject. In some embodiments, the subject is identified or diagnosed as having vasoconstriction, and the vasoconstriction in the subject is reduced after administering the dual N-type and L-type calcium channel blocker selective for the N-type calcium channel to the subject. In some embodiments, the vasoconstriction comprises vasoconstriction of a body part. In some embodiments, the temperature of the vasoconstricted body part is lower than the subject's body temperature.

In some embodiments, an increase in the temperature of a body part is measured in the subject after administration of the dual N-type and L-type calcium channel blocker selective for the N-type calcium channel. For example, an increase of at least about 0.5% (e.g., at least about 1%, 2%, 3%, 4%, 5%, 8%, 10%, 12%, 15%, or 20%) in the temperature of the body part is measured in the subject after administration of the dual N-type and L-type calcium channel blocker selective for the N-type calcium channel. In some embodiments, the increase in the temperature of the body part is an increase in one measurement or the average of a plurality of measurements taken after administration of the dual N-type and L-type calcium channel blocker selective for the N-type calcium channel relative to one measurement or the average of a plurality of measurements taken before administration. In some embodiments, when a plurality of measurements is taken before and after administration, they are taken over the same period of time (e.g., the same number of days). In some embodiments, the increase in temperature is measured by thermography. In some embodiments, the body part is a finger (e.g., an index finger, middle finger, or ring finger (e.g., an index finger)).

In some embodiments, the subject has or is being treated for Raynaud's syndrome, lupus, scleroderma, scleroderma with interstitial lung disease, rheumatoid arthritis, atherosclerosis, cryoglobulinemia, polycythemia, dermatomyositis, polymyositis, Sjögren's syndrome, or any combination thereof. In some embodiments, the treatment for lupus, scleroderma, scleroderma with interstitial lung disease, rheumatoid arthritis, atherosclerosis, cryoglobulinemia, polycythemia, dermatomyositis, polymyositis, Sjögren's syndrome, or any combination thereof comprises administering a therapeutic agent. Therapeutic agents known in the art for treating lupus, scleroderma, scleroderma with interstitial lung disease, rheumatoid arthritis, atherosclerosis, cryoglobulinemia, polycythemia, dermatomyositis, polymyositis, and Sjögren's syndrome can be found in, e.g., the Physicians' desk reference. (71st ed.). (2017). Montvale, NJ: PDR Network.

In some embodiments, the subject has scleroderma. In some embodiments, the scleroderma is limited scleroderma. In some embodiments, the scleroderma is diffuse scleroderma.

In some embodiments, the subject has (e.g., is identified or diagnosed as having) Raynaud's syndrome. In some embodiments, the Raynaud's syndrome is selected from the group consisting of: primary Raynaud's syndrome; secondary Raynaud's syndrome; Raynaud's syndrome of the nipple, nose, ear, penis, tongue, and/or any alar circulatory region. In some embodiments, the Raynaud's syndrome is primary Raynaud's syndrome. In some embodiments, the Raynaud's syndrome is secondary Raynaud's syndrome.

In some embodiments, after administration of the dual N-type and L-type calcium channel blocker selective for the N-type calcium channel one or more symptoms of the Raynaud's syndrome are improved.

In some embodiments, the symptoms are selected from the group consisting of: pain, anemia, fatigue, change in coloration of the skin, cyanosis, reperfusion, deoxygenation of the blood, digital ulcerations, reduced temperature in one or more parts of the body, changes in the endothelium of a blood vessel, swelling, impaired vision, or any combination thereof. In some embodiments, the symptom is pain.

In some embodiments, the subject has scleroderma with interstitial lung disease. In some embodiments, the method further comprises administering an agent selected from the group consisting of: a calcineurin inhibitor, cyclophosphamide, nintedanib, methotrexate, mycophenolate, a glucocorticoid (e.g., prednisone, dexamethasone, and hydrocortisone), a non-steroidal anti-inflammatory drug (e.g., aspirin, ibuprofen, or naproxen), D-penicillamine, a diuretic, omeprazole, bosentan, epoprostenol, enalapril, Lisinopril, captopril, or any combination thereof. For example, the method further comprises administering nintedanib. In some embodiments, the method further comprises administering a calcineurin inhibitor, a non-steroidal anti-inflammatory drug, or both. In some embodiments, the dual N-type and L-type calcium channel blocker selective for the N-type calcium channel, the calcineurin inhibitor, and the non-steroidal anti-inflammatory drug are administered separately, sequentially, or simultaneously. In some embodiments, the dual N-type and L-type calcium channel blocker selective for the N-type calcium channel, the calcineurin inhibitor, and the non-steroidal anti-inflammatory drug are administered separately. In some embodiments, the dual N-type and L-type calcium channel blocker selective for the N-type calcium channel, the calcineurin inhibitor, and the non-steroidal anti-inflammatory drug are administered sequentially. In some embodiments, the dual N-type and L-type calcium channel blocker selective for the N-type calcium channel, the calcineurin inhibitor, and the non-steroidal anti-inflammatory drug are administered simultaneously. In some embodiments, the dual N-type and L-type calcium channel blocker selective for the N-type calcium channel, the calcineurin inhibitor, and the non-steroidal anti-inflammatory drug are administered simultaneously as a fixed dosage form. In some embodiments, the calcineurin inhibitor is a cyclosporine. In some embodiments, the non-steroidal anti-inflammatory drug is aspirin.

In some embodiments, the subject has lupus (e.g., systemic lupus erythematosus (SLE)). In some embodiments, the method further comprises administering an agent selected from 7the group consisting of: an antimalarial drug (e.g., hydroxychloroquine), a non-steroidal anti-inflammatory drug (e.g., aspirin, ibuprofen, or naproxen), belimumab, a corticosteroid (e.g., prednisone or prednisolone), an immunosuppressant (e.g., azathioprine, cyclophosphamide, methotrexate, and mycophenolate mofetil), or any combination thereof.

In some embodiments, the subject has rheumatoid arthritis. In some embodiments, the method further comprises administering an agent selected from the group consisting of: disease-modifying anti-rheumatic drugs (e.g., methotrexate or sulfasalazine), a non-steroidal anti-inflammatory drug (e.g., aspirin, ibuprofen, or naproxen), a corticosteroid (e.g., prednisone or prednisolone), a biologic (e.g., anakinra or tocilizumab), or any combination thereof.

In some embodiments, the subject has Sjögren's syndrome. In some embodiments, the method further comprises administering an agent selected from the group consisting of: plaquenil, an antimalarial drug (e.g., hydroxychloroquine), evoxac, cevimeline, infliximab, or any combination thereof.

In some embodiments, the subject has idiopathic pulmonary fibrosis. In some embodiments, the method further comprises administering an agent selected from the group consisting of: nintedanib, pirfenidone, or any combination thereof.

In some embodiments, the subject has atherosclerosis. In some of these embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)), the atherosclerosis is treated. In some embodiments, treating the atherosclerosis comprises reducing the thickness and/or mass of a plaque in an artery of the subject. In some embodiments, the subject exhibits a reduced amount of plaque deposition in a carotid artery. In some embodiments, the reduced plaque deposition is measured by ultrasound or magnetic resonance imaging.

In some embodiments, the subject is identified or diagnosed as having reduced blood flow in a digit. In some embodiments, the subject identified or diagnosed as having reduced blood flow has a digital ulcer. In some embodiments, after administration of the dual N-type and L-type calcium channel blocker selective for the N-type calcium channel, blood flow in the subject (e.g., blood flow in a digit of the subject) is increased. In some embodiments, the digital ulcer is treated. In some embodiments, treating the digital ulcer comprises healing or improving the condition of the digital ulcer.

In some embodiments, after administration of the dual N-type and L-type calcium channel blocker selective for the N-type calcium channel in the subject, burning pain, body temperature changes of the subject, hyperesthesia, changes in skin or tissue color, edema, changes in skin turgor, ruble, pallor, cyanosis, vasospasm, or a combination thereof are treated in the subject. In some embodiments, after administration of the dual N-type and L-type calcium channel blocker selective for the N-type calcium channel in the subject, burning pain, body temperature changes of the subject, thermal hyperesthesia, changes in skin or tissue color, edema, changes in skin turgor, ruble, pallor, cyanosis, vasospasm, or a combination thereof are treated in the subject. In some embodiments, after administration of the dual N-type and L-type calcium channel blocker selective for the N-type calcium channel in the subject, burning pain, body temperature changes of the subject, changes in skin or tissue color, edema, changes in skin turgor, ruble, pallor, cyanosis, vasospasm, or a combination thereof are treated in the subject.

In some embodiments, after administration of the dual N-type and L-type calcium channel blocker selective for the N-type calcium channel to the subject, the cardiac function of the subject is improved. In some embodiments, improving the cardiac function in the subject comprises improving the left ventricular function of the subject. In some embodiments, the subject has hypertension. In some embodiments, the subject does not have hypertension.

In some embodiments, the subject has hypertension; and after administration of the dual N-type and L-type calcium channel blocker selective for the N-type calcium channel to the subject, the blood pressure of the subject is decreased and the cardiac function (e.g., left ventricular function) of the subject is improved.

In some embodiments, the subject does not have hypertension; and after administration of the dual N-type and L-type calcium channel blocker selective for the N-type calcium channel to the subject, the blood pressure of the subject is not decreased and the cardiac function (e.g., left ventricular function) of the subject is improved.

In some embodiments, the subject has hypertension and osteoporosis; and after administration of the dual N-type and L-type calcium channel blocker selective for the N-type calcium channel to the subject, the blood pressure of the subject is decreased and the bone density in the subject is increased.

In some embodiments, the subject does not have hypertension; the subject has osteoporosis; and after administration of the dual N-type and L-type calcium channel blocker selective for the N-type calcium channel to the subject, the blood pressure of the subject is not decreased and the bone density in the subject is increased.

In some embodiments, the subject has hypertension; and after administration of the dual N-type and L-type calcium channel blocker selective for the N-type calcium channel to the subject, the blood pressure of the subject is decreased and the bone density in the subject is increased.

In some embodiments, the subject does not have hypertension; and after administration of the dual N-type and L-type calcium channel blocker selective for the N-type calcium channel to the subject, the blood pressure of the subject is not decreased and the bone density in the subject is increased.

In some embodiments, the subject has hypertension and atherosclerosis; and after administration of the dual N-type and L-type calcium channel blocker selective for the N-type calcium channel to the subject, the blood pressure of the subject is decreased and the atherosclerosis in the subject is improved. In some embodiments, the subject exhibits a reduced amount of plaque deposition in a carotid artery. In some embodiments, the reduced plaque deposition is measured by ultrasound or magnetic resonance imaging.

In some embodiments, the subject does not have hypertension; the subject has atherosclerosis; and after administration of the dual N-type and L-type calcium channel blocker selective for the N-type calcium channel to the subject, the blood pressure of the subject is not decreased and the atherosclerosis in the subject is improved. In some embodiments, the subject exhibits a reduced amount of plaque deposition in a carotid artery. In some embodiments, the reduced plaque deposition is measured by ultrasound or magnetic resonance imaging.

In some embodiments, the subject has hypertension; and after administration of the dual N-type and L-type calcium channel blocker selective for the N-type calcium channel to the subject, the blood pressure of the subject is decreased and renal function in the subject is improved.

In some embodiments, the subject does not have hypertension; and after administration of the dual N-type and L-type calcium channel blocker selective for the N-type calcium channel to the subject, the blood pressure of the subject is not decreased and renal function in the subject is improved.

In some embodiments, the subject has hypertension; and after administration of the dual N-type and L-type calcium channel blocker selective for the N-type calcium channel to the subject, the blood pressure of the subject is decreased and renal function in the subject is improved. In some embodiments, the subject does not have hypertension; and after administration of the dual N-type and L-type calcium channel blocker selective for the N-type calcium channel to the subject, the blood pressure of the subject is not decreased and renal function in the subject is improved.

In some embodiments, the subject has hypertension; the subject was previously treated with antihypertensive agents before administration of the dual N-type and L-type calcium channel blocker selective for the N-type calcium channel; and after administration of the dual N-type and L-type calcium channel blocker selective for the N-type calcium channel to the subject, the blood pressure of the subject is decreased.

In some embodiments, the subject does not have hypertension; the subject was previously treated with antihypertensive agents before administration of the dual N-type and L-type calcium channel blocker selective for the N-type calcium channel; and after administration of the dual N-type and L-type calcium channel blocker selective for the N-type calcium channel to the subject, the blood pressure of the subject is is not decreased.

In some embodiments, the subject has hypertension; the subject has scleroderma; the subject has a digital ulcer; and after administration of the dual N-type and L-type calcium channel blocker selective for the N-type calcium channel to the subject, the blood pressure of the subject is decreased, and the digital ulcer is treated. In some embodiments, treating the digital ulcer comprises healing or improving the condition of the digital ulcer.

In some embodiments, the subject does not have hypertension; the subject has scleroderma; the subject has a digital ulcer; and after administration of the dual N-type and L-type calcium channel blocker selective for the N-type calcium channel to the subject, the blood pressure of the subject is not reduced, and the digital ulcer is treated. In some embodiments, treating the digital ulcer comprises healing or improving the condition of the digital ulcer.

In some embodiments, after administration of the dual N-type and L-type calcium channel blocker selective for the N-type calcium channel in the subject, norepinephrine and metabolites thereof (e.g., norepinephrine) are reduced in the subject. In some embodiments, after administration of the dual N-type and L-type calcium channel blocker selective for the N-type calcium channel in the subject, norepinephrine is reduced in the subject. In some embodiments, after administration of the dual N-type and L-type calcium channel blocker selective for the N-type calcium channel in the subject, circulating plasma concentration of norepinephrine and metabolites thereof are reduced in the subject.

In some embodiments, the dual N-type and L-type calcium channel blocker selective for the N-type calcium channel exhibits a greater selectivity for the N-type calcium channel over the L-type calcium channel than a non-N-selective calcium channel blocker.

In some embodiments, the therapeutically effective amount of the dual N-type and L-type calcium channel blocker selective for the N-type calcium channel is at least 10% lower than the therapeutically effective amount of the non-N-selective calcium channel blocker. For example, the therapeutically effective amount of the dual N-type and L-type calcium channel blocker selective for the N-type calcium channel is at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, or at least 95% lower than the therapeutically effective amount of the non-N-selective calcium channel blocker.

In some embodiments, the therapeutically effective amount of the dual N-type and L-type calcium channel blocker selective for the N-type calcium channel decreases the blood pressure (e.g., the systolic blood pressure) of the subject to a lesser degree than the therapeutically effective amount of the non-N-selective calcium channel blocker. In some embodiments, the dual N-type and L-type calcium channel blocker selective for the N-type calcium channel decreases the blood pressure (e.g., the systolic blood pressure) of the subject at least 5% less than the non-N-selective calcium channel blocker. For example, the dual N-type and L-type calcium channel blocker selective for the N-type calcium channel decreases the blood pressure (e.g., the systolic blood pressure) of the subject at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, or at least 95% less, than the non-N-selective calcium channel blocker.

In some embodiments, following the administration of the therapeutically effective amount of the dual N-type and L-type calcium channel blocker selective for the N-type calcium channel, the subject experiences reduced hypotension, lower extremity edema, and/or headache than a subject administered a therapeutically effective amount of a non-N selective calcium channel blocker. In some embodiments, the hypotension is arterial hypotension.

In some embodiments, after administration of the dual N-type and L-type calcium channel blocker selective for the N-type calcium channel, one or more (e.g., 1, 2, 3, or 4) independently selected adverse effects in the subject are ameliorated. In some embodiments, the adverse effect is selected from pain (e.g., burning pain), swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, bone weakening, joint damage or abnormalities, cold skin, red skin, lack of sensation, edema, vasoconstriction, or poor motor skills or any combination thereof are reduced in the subject. In some embodiments, changes in skin temperature, changes in skin texture, changes in skin color, changes in growth rate of hair and/or nails are understood to be abnormal changes as deemed by a medical professional. In some embodiments, the adverse effect is selected from pain (e.g., burning pain), sensitivity to cold, changes in skin temperature, changes in skin texture, changes in skin color, changes in growth rate of hair and/or nails, or any combination thereof. In some embodiments, the adverse effect is pain (e.g., burning pain). In some embodiments, adverse effect is sensitivity to cold. In some embodiments, adverse effect is changes in skin temperature. In some embodiments, adverse effect is changes in skin texture. In some embodiments, adverse effect is changes in skin color. In some embodiments, adverse effect is changes in growth rate of hair and/or nails. In some embodiments, the pain is neuropathic pain. In some embodiments, the pain is selected from burning pain, hyperpathia, hyperalgesia, and allodynia (e.g., thermal allodynia). In some embodiments, the pain is selected from burning pain, hyperpathia, and thermal allodynia. In some embodiments, the pain is burning pain. In some embodiments, the pain is chronic pain. In some embodiments, the pain intensifies upon exposure of the subject to stress, sound, or light; or when the subject exercises.

In some embodiments, the adverse effect is vasoconstriction. In some embodiments, the vasoconstriction comprises vasoconstriction of a body part, and the temperature of the vasoconstricted body part is lower than the subject's body temperature. In certain of these embodiments, the dual N-type and L-type calcium channel blocker selective for the N-type calcium channel increases the temperature of the vasoconstricted body part to the subject's body temperature at least 5% faster than the non-N-selective calcium channel blocker. For example, the dual N-type and L-type calcium channel blocker selective for the N-type calcium channel increases the temperature of the vasoconstricted body part to the subject's body temperature at least 10% faster, at least 15% faster, at least 20% faster, at least 25% faster, at least 30% faster, at least 35% faster, at least 40% faster, at least 45% faster, at least 50% faster, at least 55% faster, at least 60% faster, at least 65% faster, at least 70% faster, at least 75% faster, at least 80% faster, at least 85% faster, at least 90% faster, or at least 95% faster than the non-N-selective calcium channel blocker. In some embodiments, the difference can be measured in seconds or minutes.

In some embodiments, the dual N-type and L-type calcium channel blocker selective for the N-type calcium channel is more effective than the non-N-selective calcium channel blocker in treating an adverse effect of the complex regional pain syndrome. In some embodiments, the adverse effect is a symptom or a clinical manifestation of the complex regional pain syndrome. In some embodiments, the subject experiences less frequent, less severe, and/or shorter episodes of adverse events than when administered a therapeutically effective amount of a non-N-selective calcium channel blocker useful to treat the complex regional pain syndrome. In some embodiments, the adverse events are one or more events selected from the group consisting of: pain (e.g., burning pain), swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, bone weakening, joint damage or abnormalities, cold skin, red skin, lack of sensation, edema, vasoconstriction, or poor motor skills or any combination thereof are reduced in the subject. In some embodiments, the pain is neuropathic pain. In some embodiments, the pain is selected from burning pain, hyperpathia, hyperalgesia, and allodynia (e.g., thermal allodynia). In some embodiments, the pain is selected from burning pain, hyperpathia, and thermal allodynia. In some embodiments, the pain is burning pain. In some embodiments, the pain is chronic pain. In some embodiments, the pain intensifies upon exposure of the subject to stress, sound, or light; or when the subject exercises. In some embodiments, the subject experiences less frequent, less severe, and/or shorter episodes of pain (e.g., neuropathic pain (e.g., burning pain)) than when administered a therapeutically effective amount of a non-N-selective calcium channel blocker useful to treat the complex regional pain syndrome.

In some embodiments, the therapeutically effective amount of the dual N-type and L-type calcium channel blocker selective for the N-type calcium channel is reduced compared to the therapeutically effective amount of a non-N-selective calcium channel blocker useful to treat the complex regional pain syndrome.

In some embodiments, one or more side effects experienced by the subject after administration of the dual N-type and L-type calcium channel blocker selective for the N-type calcium channel are less severe or less frequent than as compared to the side effects experienced by a subject after administration of a therapeutically effective amount of a non-N-selective calcium channel blocker useful to treat the disease or disorder. Without wishing to be bound by theory, this may allow a higher dose of the dual N-type and L-type calcium channel blocker selective for the N-type calcium channel to be administered to the subject, which can, e.g., result in a higher treatment efficacy than the non-N-selective calcium channel blocker. In some embodiments, the side effects are selected from dizziness, peripheral edema, lower extremity edema, flushing, flushing sensation, acute myocardial infarction, muscle cramps, tremor, cough, dyspnea, hypotension, wheezing, and increased gastroesophageal reflux.

It has been shown that dual N-type and L-type calcium channel blockers selective for the N-type calcium channels have fewer and less severe side effects, better tolerability, and are safer than non-N-selective calcium channel blockers. It is believed that this is due to the increased inhibition of the N channel relative to the L channel. By decreasing sympathetic activity, as well as by dilating not only arterioles but the venous system, dual N-type and L-type calcium channel blockers selective for the N-type calcium channel appear to be associated with less adverse events in patients treated for hypertension than patients treated with dual L and N-calcium channel antagonists with lower levels of N-selectivity.

It is understood that dual N-type and L-type calcium channel blocker selective for the N-type calcium channel may decrease the blood pressure of subjects that are hypertensive. As such, it may be beneficial to administer an agent that increases blood pressure in combination with the dual N-type and L-type calcium channel blocker selective for the N-type calcium channel. In some embodiments of the methods disclosed herein, the method further comprises administering to the subject a therapeutically effective amount of an agent that increases blood pressure. In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines. In some embodiments, the blood pressure of the subject before and after administration of the dual N-type and L-type selective calcium blocker and the agent that increases blood pressure is substantially the same. In some other embodiments, the blood pressure of the subject after administration of the dual N-type and L-type selective calcium blocker and the agent that increases blood pressure is less than 20% (e.g., less than 15%, less than 10%, less than 5%, less than 3% or less than 1%) higher or lower than the blood pressure of the subject before administration of the dual N-type and L-type selective calcium blocker and the agent that increases blood pressure.

In some embodiments, the treating comprises reducing pulmonary hypertension in the subject.

In some embodiments, the subject is also diagnosed with hypertension; and wherein after administration of the dual N-type and L-type calcium channel blocker selective for the N-type calcium channel to the subject, the blood pressure (e.g., systolic blood pressure) of the subject is reduced. In some embodiments, the subject was not diagnosed with hypertension; and wherein after administration of the dual N-type and L-type calcium channel blocker selective for the N-type calcium channel to the subject, the blood pressure (e.g., the systolic blood pressure) of the subject is not reduced. Without wishing to be bound by theory, it is believed that when the subject has hypertension, the dual N-type and L-type calcium channel blocker selective for the N-type calcium channel reduces the blood pressure of the subject; however, when the subject does not have hypertension (i.e., the subject is normotensive), the dual N-type and L-type calcium channel blocker selective for the N-type calcium channel does not reduce the blood pressure of the subject.

In some embodiments, the systolic blood pressure of the subject is reduced by greater than about 1 mm Hg (e.g., greater than about 2 mm Hg, greater than about 5 mm Hg, greater than about 10 mm Hg, greater than about 15 mm Hg, greater than about 20 mm Hg, greater than about 30 mm Hg, or greater than about 40 mm Hg). In some embodiments, the systolic blood pressure of the subject is reduced by greater than 10 mm Hg.

In some embodiments, the subject is identified as normotensive; and wherein after administration of the dual N-type and L-type calcium channel blocker selective for the N-type calcium channel to the subject, the systolic blood pressure of the subject changes by less than 20% (e.g., less than 18%, less than 16%, less than 14%, less than 12%, less than 10%, less than 8%, less than 6%, less than 5%, less than 4%, less than 3%, less than 2%, or less than 1%). In some embodiments, the subject is identified as normotensive; and wherein after administration of the dual N-type and L-type calcium channel blocker selective for the N-type calcium channel to the subject, the systolic blood pressure of the subject changes by less than 5%. In some embodiments, the subject is identified as normotensive; and wherein after administration of the dual N-type and L-type calcium channel blocker selective for the N-type calcium channel to the subject, the systolic blood pressure of the subject remains substantially the same. In some embodiments, the subject is identified as normotensive; and wherein after administration of the dual N-type and L-type calcium channel blocker selective for the N-type calcium channel to the subject, the heart rate and systolic blood pressure of the subject remains substantially the same.

In some embodiments, after administration of the dual N-type and L-type calcium channel blocker selective for the N-type calcium channel, the bone density of the subject does not decrease. In some of these embodiments, the bone density of the subject increases. This may occur through a reduction in the number of osteoclasts in the subject and/or an increase in the ratio of alkaline phosphate to tartrate resistant acid phosphatase (TRAP).

In some embodiments, the method further comprises selecting a subject identified or diagnosed as having reduced bone density for the treatment. In some embodiments, the subject identified or diagnosed as having reduced bone density has osteoporosis. In some embodiments, the subject is female.

In some embodiments, the method further comprises selecting a subject identified or diagnosed as having reduced renal function for the treatment. In some embodiments, the renal function of the subject is not reduced after treatment. In some embodiments, the renal function of the subject is improved after treatment.

In some embodiments, improving renal function comprises determining one or more of: a reduction in intrarenal arterial stiffness, improved blood flow to the kidneys, increased expression levels of podocyte proteins, reduction in urinary protein excretion, improvement in glomerular filtration rate, reduction in plasma creatinine, decrease in brachial-ankle pulse wave velocity, improvement in plasma inulin clearance, or any combination thereof. In some embodiments, improved renal function comprises a reduction in intrarenal arterial stiffness, improved blood flow to the kidneys, increased expression levels of podocyte proteins, or any combination thereof.

In some embodiments, after administration of the dual N-type and L-type calcium channel blocker selective for the N-type calcium channel, sympathetic tone diminution, direct smooth muscle relaxation, dysesthetic pain, burning pain, body temperature changes of the subject, hyperesthesia, changes in skin or tissue color, edema, changes in skin turgor, ruble, pallor, cyanosis, vasospasm, or any combination thereof are treated in the subject. In some embodiments, after administration of the dual N-type and L-type calcium channel blocker selective for the N-type calcium channel, sympathetic tone diminution, direct smooth muscle relaxation, dysesthetic pain, burning pain, body temperature changes of the subject, changes in skin or tissue color, edema, changes in skin turgor, ruble, pallor, cyanosis, vasospasm, or any combination thereof are treated in the subject. In some embodiments, after administration of the dual N-type and L-type calcium channel blocker selective for the N-type calcium channel, a reduction in sympathetic tone diminution, direct smooth muscle relaxation, dysesthetic pain, burning pain, body temperature changes of the subject, hyperesthesia, changes in skin or tissue color, edema, changes in skin turgor, ruble, pallor, cyanosis, vasospasm, or any combination thereof is observed in the subject. In some embodiments, after administration of the dual N-type and L-type calcium channel blocker selective for the N-type calcium channel, a reduction in sympathetic tone diminution, direct smooth muscle relaxation, dysesthetic pain, burning pain, body temperature changes of the subject, changes in skin or tissue color, edema, changes in skin turgor, ruble, pallor, cyanosis, vasospasm, or any combination thereof is observed in the subject.

In some embodiments, after administration of the dual N-type and L-type calcium channel blocker selective for the N-type calcium channel to the subject, nitric oxide is increased in the subject.

In some embodiments, after administration of the dual N-type and L-type calcium channel blocker selective for the N-type calcium channel to the subject, nitric oxide is not increased in the subject.

In some embodiments, after administration of the dual N-type and L-type calcium channel blocker selective for the N-type calcium channel endothelial dysfunction in the subject is improved.

In some embodiments, after administration of the dual N-type and L-type calcium channel blocker selective for the N-type calcium channel oxidative stress in the subject is decreased. In some embodiments, decreasing oxidative stress in the subject comprises decreasing oxidative stress in the subject after administration of the dual N-type and L-type calcium channel blocker selective for the N-type calcium channel relative to oxidative stress in the subject before administration of the dual N-type and L-type selective calcium blocker.

In some embodiments, an antioxidant selected from the group consisting of a hydralazine compound, a glutathione, vitamin C, cysteine, β-carotene, a ubiquinone, a ubiquinol-10, a tocopherol, coenzyme Q, or a mixture thereof is not administered to the subject. In some embodiments, an anti-oxidant is not administered to the subject.

In some embodiments, the subject is a mammal. In some embodiments, the subject is human. In some embodiments, the subject is female. In some embodiments, the subject is male.

In some embodiments, the dual N-type and L-type calcium channel blocker selective for the N-type calcium channel exhibits at least a 2-fold selectivity (e.g., at least a 4-fold, 10-fold, 20-fold, 30-fold, 40-fold, 50-fold, 60-fold, 80-fold, 100-fold, 130-fold, 150-fold, or 200-fold selectivity) for the N-type calcium channel over an L-type calcium channel. In some embodiments, the dual N-type and L-type calcium channel blocker selective for the N-type calcium channel exhibits at least a 50-fold selectivity for the N-type calcium channel over an L-type calcium channel. In some embodiments, the dual N-type and L-type calcium channel blocker selective for the N-type calcium channel exhibits a 50-fold to 100-fold selectivity for the N-type calcium channel over an L-type calcium channel.

In some embodiments, the dual N-type and L-type calcium channel blocker selective for the N-type calcium channel exhibits a greater selectivity for the N-type calcium channel over the L-type calcium channel than a non-N-selective calcium channel blocker. In some embodiments, the dual N-type and L-type calcium channel blocker selective for the N-type calcium channel exhibits at least a 5% (e.g., at least a 10%, 20%, 30%, 40%, 50%, 60%, 70%, 80%, 100%, 150%, 200%, 300%) greater selectivity for the N-type calcium channel over the L-type calcium channel than a non-N-selective calcium channel blocker.

In some embodiments, the dual N-type and L-type calcium channel blocker selective for the N-type calcium channel is selected from the group consisting of: cilnidipine, Z-160, CNV2197944, or pharmaceutically acceptable salts thereof. In some embodiments, the dual N-type and L-type calcium channel blocker selective for the N-type calcium channel is cilnidipine or a pharmaceutically acceptable salt thereof.

In some embodiments, the method further comprises administering a therapeutically effective amount of a phosphodiesterase type 5 inhibitor to the subject. In some embodiments, the phosphodiesterase type 5 inhibitor is selected from sildenafil, tadalafil, or pharmaceutically acceptable salts thereof. In some embodiments, the phosphodiesterase type 5 inhibitor is tadalafil or a pharmaceutically acceptable salt thereof.

In some embodiments, the method comprises administering at least one additional therapeutic agent to the subject. The at least one additional therapeutic agent can be administered simultaneously, separately, sequentially, or in combination (e.g., for more than two agents) with the dual N-type and L-type calcium channel blocker selective for the N-type calcium channel. Non-limiting examples of additional therapeutic agents include calcium channel blockers, sodium channel blockers (e.g., Nav 1.7 sodium channel blocker), TRP-v1 inhibitors, and therapeutic agents that relieve pain.

In some embodiments, the at least one additional therapeutic agent is selected from the group consisting of: analgesics (e.g., acetaminophen), anesthetics, anticonvulsants, antidepressants, oral muscle relaxants, corticosteroids, calcitonin, bisphosphonates (e.g., neridronate), cyclooxygenase (COX)-2 inhibitors, free-radical scavenger agents (e.g., ketoprofen), prednisolone, prednisone, oral steroids, opioids, riociguat, amlodipine, pregabalin, alendronate, pamidronate, gabapentin and gabapentenoids, nifedipine, nicardipine, conotoxins, cadmium, bupivacaine, epinephrine, caroverine, levetiracetam, lamotrigine, NP078585, TROX-1, non-steroidal anti-inflammatory agents (e.g., ibuprofen), ascorbic acid (Vitamin C), valsartan, dimethylsulfoxide, N-acetylcysteine, phenoxybenzamine.

In some embodiments, the at least one additional therapeutic agent is selected from the group consisting of: riociguat, amlodipine, pregabalin, gabapentin, nifedipine, nicardipine, conotoxins, cadmium, caroverine, levetiracetam, lamotrigine, NP078585, pregabalin, TROX-1, acetaminophen, non-steroidal anti-inflammatory agents (e.g., ibuprofen), valsartan, dimethylsulfoxide, N-acetylcysteine, vitamin C, N-acetyl cysteine, 4-hydroxy-2,2,6,6-tetramethylpiperidin-1-oxyl, magnesium sulfate, and ziconotide.

In some embodiments, the at least one additional therapeutic agent is selected from the group consisting of: riociguat, amlodipine, pregabalin, gabapentin, nifedipine, nicardipine, conotoxins, cadmium, caroverine, levetiracetam, lamotrigine, NP078585, pregabalin, TROX-1, non-steroidal anti-inflammatory agents (e.g., ibuprofen), valsartan, dimethylsulfoxide, N-acetylcysteine, N-acetyl cysteine, 4-hydroxy-2,2,6,6-tetramethylpiperidin-1-oxyl, magnesium sulfate, ziconotide, analgesics (e.g., acetaminophen), anesthetics, anticonvulsants, antidepressants, oral muscle relaxants, corticosteroids, calcitonin, bisphosphonates (e.g., neridronate), cyclooxygenase (COX)-2 inhibitors, free-radical scavenger agents (e.g., ketoprofen), prednisolone, prednisone, oral steroids, opioids, riociguat, amlodipine, pregabalin, alendronate, pamidronate, gabapentin, nifedipine, nicardipine, bupivacaine, epinephrine, caroverine, ascorbic acid (Vitamin C), valsartan, dimethylsulfoxide, N-acetylcysteine, phenoxybenzamine.

In some embodiments, the at least one additional therapeutic agent is selected from the group consisting of: oxycodone, tramadol, Dilaudid, OxyContin, Cymbalta, a statin, gabapentin, pregabalin, an angiotensin-converting-enzyme (ACE) inhibitor, an angiotensin receptor blocker (ARB), niacin, a proton pump inhibitor, aspirin, Fentanyl Transdermal System, acetaminophen/oxycodone, Roxicodone, Ultram, hydromorphone, Percocet, MS Contin, Butrans, morphine, hydromorphone, methadone, buprenorphine, duloxetine, fentanyl, Duragesic, Endocet, Roxanol, Kadian, Roxicet, ConZip, Methadose, Oxyfast, Dazidox, Fentora, Irenka, Methadone Diskets, Oramorph SR, Roxicodone Intensol, Xtampza ER, Actiq, Belbuca, ETH-Oxydose, Infumorph, naloxone/pentazocine, Oxaydo, Oxydose, OxyIR, ziconotide, Abstral, Astramorph PF, Buprenex, Dolophine, Duramorph, Duramorph PF, Embeda, Lazanda, MorphaBond ER, morphine/naltrexone, Prialt, RMS, Roxanol-T, Sublimaze, Subsys, Talwin Nx, Magnacet, Nalocet, Narvox, Perloxx, Primlev, Xolox, and Prolate.

In some embodiments, the additional therapeutic agent is selected from the group consisting of cilostazol, bosentan, prostacyclin analogs, Serotonin receptor antagonists and reuptake inhibitors, N-acetylcysteine, statins, botulinum toxin A, topical vitamin E, vitamin C, and combinations thereof.

In some embodiments, the additional therapeutic agent is selected from the group consisting of: an aluminum antacid, a magnesium antacid, a calcium channel blocker that is different from the dual N-type and L-type calcium channel blocker selective for the N-type calcium channel (e.g., a non-N-selective calcium channel blocker (e.g., diltiazem, amlodipine, or verapamil)), colchicine, warfarin, prednisone, a diphosphonate, and sodium etidronate.

In some aspects, disclosed herein is a method of treating complex regional pain syndrome in a subject in need thereof, the method comprising administering to the subject (a) a therapeutically effective amount of a dual N-type and L-type calcium channel blocker selective for the N-type calcium channel, and (b) at least one additional therapeutic agent.

In some aspects, disclosed herein is a method of treating complex regional pain syndrome in a subject in need thereof, the method comprising administering to the subject (a) a therapeutically effective amount of a dual N-type and L-type calcium channel blocker selective for the N-type calcium channel, and (b) an opioid.

In some embodiments, the at least one additional therapeutic agent is an opioid. In some embodiments, the opioid is selected from the group consisting of: fentanyl, heroin, hydromorphone, oxymorphone, methadone, oxycodone, morphine, hydrocodone, codeine, meperidine, tramadol, and combinations thereof. In some embodiments, the opioid is oxycodone. In some embodiments, the opioid is hydromorphone. In some embodiments, the opioid is oxymorphone. In some embodiments, the opioid is methadone. In some embodiments, the opioid is hydrocodone. In some embodiments, the opioid is meperidine. In some embodiments, the opioid is tramadol.

In some aspects, disclosed herein is a method of treating complex regional pain syndrome in a subject in need thereof, the method comprising administering to the subject (a) a therapeutically effective amount of a dual N-type and L-type calcium channel blocker selective for the N-type calcium channel, and (b) oxycodone.

In some embodiments, the dosage of the opioid (e.g., oxycodone) is from about 1 μg to about 100 mg (e.g., from about 1 μg to about 5 mg, from about 1 mg to about 50 mg, from about 1 mg to about 25 mg, from about 1 mg to about 15 mg, from about 1 mg to about 10 mg, from about 1 mg to about 5 mg, from about 1 mg to about 3 mg, from about 3 mg to about 35 mg, from about 5 mg to about 25 mg, from about 8 mg to about 28 mg, from about 12 mg to about 28 mg, from about 9 mg to about 21 mg, from about 15 mg to about 25 mg, from about 20 mg to about 30 mg, from about 22 mg to about 28 mg, from about 17 mg to about 23 mg, from about 8 mg to about 12 mg, about 8 mg, about 0.1 mg, about 0.5 mg, about 1 mg, about 2 mg, about 3 mg, about 4 mg, about 5 mg, about 6 mg, about 8 mg, about 10 mg, about 12 mg, about 18 mg, about 20 mg, about 22 mg, or about 25 mg). In some embodiments, the dosage of the opioid is from about 1 mg to about 25 mg. In some embodiments, the dosage of the opioid is from about 1 mg to about 15 mg. In some embodiments, the dosage of the opioid is about 1 mg. In some embodiments, the dosage of the opioid is about 3 mg. In some embodiments, the dosage of the opioid is about 5 mg. In some embodiments, the dosage of the opioid is about 10 mg. In some embodiments, the dosage of the opioid is about 20 mg. In some embodiments, the dosage of the opioid is about 25 mg.

In some embodiments, the treating further comprises physical therapy. In some embodiments, the treating further comprises psychotherapy and/or biofeedback therapy. In some embodiments, during or after psychotherapy and/or biofeedback therapy, one or more of anxiety, depression, and avoidant behavior associated with pain are treated or reduced in the subject. In some embodiments, the treating further comprises at least one (e.g., 1, 2, 3, or 4) sympathetic nerve block (e.g., at least one para-spinal lumbar sympathetic nerve block).

In some embodiments, the dual N-type and L-type calcium channel blocker selective for the N-type calcium channel does not include a tetravalent (i.e., quaternized) nitrogen wherein the four substituents bonded to the nitrogen are non-hydrogen substituents.

In some embodiments, the dual N-type and L-type calcium channel blocker selective for the N-type calcium channel does not include a sulfide (i.e., —S—) moiety.

In some embodiments of any method disclosed herein, the subject is not infected with SARS-COV-2. In some embodiments of any method disclosed herein, the subject is not afflicte with (i.e., not suffering from) SARS-COV-2 infection. In some embodiments of any method disclosed herein, the subject is not being treated for SARS-CoV-2. In some embodiments of any method disclosed herein, the subject is not diagnosed with SARS-COV-2 (e.g., after the subject is subjected to a test that diagnoses whether the subject is infected with SARS-COV-2 (e.g., a PCR test, an antigen test, or an antibody test)).

In some embodiments of any method disclosed herein, the subject is infected with SARS-COV-2. In some embodiments of any method disclosed herein, the subject is afflicted with (i.e., suffering from) SARS-COV-2. In some embodiments of any method disclosed herein, the subject is being treated for SARS-COV-2 infection. In some embodiments of any method disclosed herein, the subject is diagnosed with SARS-COV-2 infection (e.g., after the subject is subjected to a test that diagnoses whether the subject is infected with SARS-COV-2 (e.g., a PCR test, an antigen test, or an antibody test)).

In some embodiments, greater than about 2% (e.g., greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, or greater than about 65%, greater than about 70%, greater than about 75%, greater than about 80%, greater than about 85%, greater than about 90%, greater than about 95%, greater than about 97%, or greater than about 99%) of the dual N-type and L-type calcium channel blocker selective for the N-type calcium channel is anionic at physiological pH (e.g., a pH of from from about 7.2 to about 7.6 (e.g., about 7.4)).

In some embodiments, the method further comprises determining that the subject has sympathetic overactivity or elevated sympathetic outflow (e.g., before administration of the dual N-type and L-type calcium channel blocker selective for the N-type calcium channel to the subject). In some embodiments, the method further comprises identifying a subject having sympathetic overactivity or elevated sympathetic outflow (e.g., before administration of the dual N-type and L-type calcium channel blocker selective for the N-type calcium channel to the subject). In some embodiments, the method further comprises determining whether the subject has a clinical record indicating sympathetic overactivity or elevated sympathetic outflow (e.g., before administration of the dual N-type and L-type calcium channel blocker selective for the N-type calcium channel to the subject). In some embodiments, the method further comprises monitoring sympathetic activity or sympathetic outflow in the subject during treatment. In some embodiments, the method further comprises determining a decrease in sympathetic activity or sympathetic outflow in the subject after treatment. In some embodiments, determining, identifying, or monitoring sympathetic activity or sympathetic outflow in the subject is performed using galvanic skin testing and/or plethysmography

In some embodiments, the dual N-type and L-type calcium channel blocker selective for the N-type calcium channel further inhibits a Nav 1.7 sodium channel.

In some embodiments, following the administration of the therapeutically effective amount of the dual N-type and L-type calcium channel blocker selective for the N-type calcium channel, the subject has an increase in Nav 1.7 inhibition. In some embodiments, the Nav 1.7 inhibition comprises inhibition of the closed state of the Nav 1.7 sodium channel. In some embodiments, the Nav 1.7 inhibition comprises inhibition of the inactivated state of the Nav 1.7 sodium channel. In some embodiments, the inhibition of the closed state of the Nav 1.7 sodium channel is greater than the inhibition of the inactivated state of the Nav 1.7 sodium channel. In some embodiments, the inhibition of the inactivated state of the Nav 1.7 sodium channel is greater than inhibition of the closed state of the Nav 1.7 sodium channel.

In some embodiments, following the administration of the therapeutically effective amount of the dual N-type and L-type calcium channel blocker selective for the N-type calcium channel, the subject has an at least 1% increase in Nav 1.7 inhibition. For example, the subject has an at least 5%, at least 10%, at least 20%, at least 30%, at least 40%, at least 50%, at least 60%, at least 70%, at least 80%, at least 90%, or at least 95% increase in Nav 1.7 inhibition. In some embodiments, following the administration of the therapeutically effective amount of the Nav 1.7 sodium channel blocker, the subject has an at least 10% increase in Nav 1.7 inhibition. In some embodiments, following the administration of the therapeutically effective amount of the Nav 1.7 sodium channel blocker, the subject has an at least 20% increase in Nav 1.7 inhibition. In some embodiments, following the administration of the therapeutically effective amount of the Nav 1.7 sodium channel blocker, the subject has an at least 15% increase in Nav 1.7 inhibition. In some embodiments, following the administration of the therapeutically effective amount of the Nav 1.7 sodium channel blocker, the subject has an at least 35% increase in Nav 1.7 inhibition.

In some embodiments, the dual N-type and L-type calcium channel blocker selective for the N-type calcium channel further inhibits a TRP-v1 channel.

In some embodiments, the method further comprises determining that the subject has TRP-v1 overactivation (e.g., before administration of the dual N-type and L-type calcium channel blocker selective for the N-type calcium channel to the subject). In some embodiments, the method further comprises identifying a subject having TRP-v1 overactivation (e.g., before administration of the dual N-type and L-type calcium channel blocker selective for the N-type calcium channel to the subject). In some embodiments, the method further comprises determining whether the subject has a clinical record indicating or showing TRP-v1 overactivation (e.g., before administration of the dual N-type and L-type calcium channel blocker selective for the N-type calcium channel to the subject). In some embodiments, the method further comprises monitoring TRP-v1 overactivation in the subject during treatment. In some embodiments, the method further comprises determining a decrease in TRP-v1 activation or upregulation in the subject after treatment. In some embodiments, determining that the subject has TRPv-1 overactivation comprises determining an abnormally high TRP-v1 current density and capsaicin responding rate in small-sized nociceptive dorsal root ganglion (DRG) neurons. Methods for determining TRP-v1 overactivation in a subject include methods disclosed in Nature Reviews Drug Discovery, 2022, 21, 41-59.

In some embodiments, following the administration of the therapeutically effective amount of the dual N-type and L-type calcium channel blocker selective for the N-type calcium channel, the subject has an at least 1% increase in TRP-v1 inhibition. For example, the subject has an at least 5%, at least 10%, at least 20%, at least 30%, at least 40%, at least 50%, at least 60%, at least 70%, at least 80%, at least 90%, or at least 95% increase in TRP-v1 inhibition. In some embodiments, following the administration of the therapeutically effective amount of the dual N-type and L-type calcium channel blocker selective for the N-type calcium channel, the subject has an at least 10% increase in TRP-v1 inhibition. In some embodiments, following the administration of the therapeutically effective amount of the dual N-type and L-type calcium channel blocker selective for the N-type calcium channel, the subject has an at least 20% increase in TRP-v1 inhibition. In some embodiments, following the administration of the therapeutically effective amount of the dual N-type and L-type calcium channel blocker selective for the N-type calcium channel, the subject has an at least 15% increase in TRP-v1 inhibition. In some embodiments, following the administration of the therapeutically effective amount of the dual N-type and L-type calcium channel blocker selective for the N-type calcium channel, the subject has an at least 35% increase in TRP-v1 inhibition. In some embodiments, measuring the increase in TRP-v1 inhibition comprises measuring a decrease in TRP-v1 protein expression by immunofluorescence staining. See, for example, Front Pharmacol. 2019; 10:453, which is incorporated by reference herein in its entirety.

In some embodiments, the method further comprises determining an abnormally high inflammation in the subject before administration of the dual N-type and L-type calcium channel blocker selective for the N-type calcium channel. In some embodiments, the method further comprises determining a reduction in inflammation in the subject after administration of the dual N-type and L-type calcium channel blocker selective for the N-type calcium channel. In some embodiments, the inflammation is generalized, neurogenic, or both.

In some embodiments, the method further comprises determining a reduced concentration of NF-κB in the subject after administration of the dual N-type and L-type calcium channel blocker selective for the N-type calcium channel. In some embodiments, the concentration of NF-κB is measured in the the plasma of the subject.

In some embodiments, the method further comprises determining an increase in expression of phosphatidylinositol 3-kinase, phosphorylated Akt, phosphorylated glycogen synthase kinase-3 (pGSK-3b), heat shock transcription factor (HSTF-1), or any combination thereof in the subject after administration of the dual N-type and L-type calcium channel blocker selective for the N-type calcium channel.

In some embodiments, the method further comprises determining a decrease in expression of cytosolic cytochrome c, activated caspase 3, cleaved poly(ADP-ribose) polymerase (PARP), or any combination thereof in the subject after administration of the dual N-type and L-type calcium channel blocker selective for the N-type calcium channel.

In some embodiments, the method further comprises determining a reduced concentration of catecholamine, aldosterone, brain natriuretic peptide, urine liver-type fatty acid binding protein, albumin excretion ratio, or any combination thereof in the subject after administration of the dual N-type and L-type calcium channel blocker selective for the N-type calcium channel. In some embodiments, the concentration of concentration of catecholamine, aldosterone, brain natriuretic peptide, urine liver-type fatty acid binding protein, and albumin excretion ratio are measured in the the plasma of the subject.

In some embodiment, the method further comprises determining that the subject has dysregulation of ERK1 and/or ERK2 before administration of the dual N-type and L-type calcium channel blocker selective for the N-type calcium channel. In some embodiments, the method further comprises determining inhibition or downregulation of ERK1 and/or ERK2 in the subject after administration of the dual N-type and L-type calcium channel blocker selective for the N-type calcium channel.

In some embodiments, the method further comprises determining that the subject has upregulation of or abnormally elevated concentration (e.g., abnormally elevated concentration) of TNF-α, IL-1B. IL2, IL-6, ET-I, cGRP, bradykinin, substance P, MMP9, CXCL13, or any combination thereof before administration of the dual N-type and L-type calcium channel blocker selective for the N-type calcium channel. In some embodiments, the method further comprises determining a lower concentration of TNF-α, IL-1B, IL2, IL-6, ET-I, cGRP, bradykinin, substance P, MMP9, CXCL13, or any combination thereof in the subject after administration of the dual N-type and L-type calcium channel blocker selective for the N-type calcium channel. In some embodiments, the concentration of TNF-α, IL-1B. IL2, IL-6, ET-I, cGRP, bradykinin, substance P, MMP9, CXCL13, are measured in the cerebrospinal fluid or the plasma (e.g., the plasma) of the subject.

In some embodiments, the method further comprises determining that the subject has abnormally low concentration of IL-8 mRNA, IL-4, IL-10, or any combination thereof before administration of the dual N-type and L-type calcium channel blocker selective for the N-type calcium channel. In some embodiments, the method further comprises determining that the concentration of IL-8 mRNA, IL-4, IL-10, or any combination thereof increased in the subject after administration of the dual N-type and L-type calcium channel blocker selective for the N-type calcium channel. In some embodiments, the concentration of IL-8 mRNA, IL-4, IL-10, or any combination thereof are measured in the cerebrospinal fluid or the plasma (e.g., the plasma) of the subject.

In some embodiments, the method further comprises determining that the subject has an abnormally high immunologic response, an abnormally high number or concentration of non-specific autoantibodies to sympathetic receptors, or both before administration of the dual N-type and L-type calcium channel blocker selective for the N-type calcium channel. In some embodiments, the method further comprises determining a reduction in immunologic response, a reduction in number or concentration of non-specific autoantibodies to sympathetic receptors, or both after administration of the dual N-type and L-type calcium channel blocker selective for the N-type calcium channel.

In some embodiments, the method further comprises determining autonomic dysregulation; an abnormally high number of sympathetic receptors; an abnormally high concentration of circulating catecholamines; sympathetic hyperactivity; or any combination thereof in the subject before administration of the dual N-type and L-type calcium channel blocker selective for the N-type calcium channel. In some embodiments, the method further comprises determining an improvement in autonomic functioning; a reduction in sympathetic receptors; a reduction in the concentration of circulating catecholamines; a reduction in sympathetic activity; or any combination thereof in the subject after administration of the dual N-type and L-type calcium channel blocker selective for the N-type calcium channel.

In some embodiments, the method further comprises determining an abnormally high central sensitization and neuroplasticity with increased substance P, bradykinin, and/or glutamate; an overexpression of spinal NMDA receptors; gabaminergic overactivity; or any combination thereof in the subject before administration of the dual N-type and L-type calcium channel blocker selective for the N-type calcium channel. In some embodiments, the method further comprises determining a reduction in central sensitization and neuroplasticity with increased substance P, bradykinin, and/or glutamate; an overexpression of spinal NMDA receptors; gabaminergic overactivity; or any combination thereof in the subject after administration of the dual N-type and L-type calcium channel blocker selective for the N-type calcium channel.

In some embodiments, the method further comprises determining peripheral hypersensitization; an abnormally high concentration of spinal N-methyl-D-aspartate; an abnormally high concentration of spinal glutamate; upregulation in TNF-α and/or TRP-V1 activation; or any combination thereof in the subject before administration of the dual N-type and L-type calcium channel blocker selective for the N-type calcium channel. In some embodiments, the method further comprises determining a reduction in peripheral sensitization; a reduction in spinal N-methyl-D-aspartate; a reduction in spinal glutamate; a reduction in upregulation of TNF-α and/or TRP-V1 activation; or any combination thereof in the subject after administration of the dual N-type and L-type calcium channel blocker selective for the N-type calcium channel.

In some embodiments, the method further comprises determining abnormally high oxidative stress, a reduction in one or more oxidative stress markers, an abnormally high concentration of reactive oxygen species, immune system overactivation, or any combination thereof in the subject before administration of the dual N-type and L-type calcium channel blocker selective for the N-type calcium channel. In some embodiments, the method further comprises determining a reduction in oxidative stress, a reduction in one or more oxidative stress markers, a reduction in reactive oxygen species, a reduction in immune system activation, or any combination thereof in the subject after administration of the dual N-type and L-type calcium channel blocker selective for the N-type calcium channel. In some embodiments, the one or more oxidative stress markers are selected from nitrotyrosine, isoprostane, lactic dehydrogenase, uric acid, malondialdehyde, myeloperoxidase, oxidized low density lipoproteins, and S-glutathionylation of haemoglobin.

In some embodiments, the method further comprises determining that the subject has microvascular pathology and/or ischemia reperfusion injury before administration of the dual N-type and L-type calcium channel blocker selective for the N-type calcium channel. In some embodiments, the method further comprises determining that the microvascular pathology and/or ischemia reperfusion injury in the subject is treated, improved, or ameliorated after administration of the dual N-type and L-type calcium channel blocker selective for the N-type calcium channel.

In some embodiments, the method further comprises determining endothelial dysfunction in the subject before administration of the dual N-type and L-type calcium channel blocker selective for the N-type calcium channel. In some embodiments, endothelial function in the subject is improved after administration of the dual N-type and L-type calcium channel blocker selective for the N-type calcium channel.

In some embodiments, the method further comprises determining an abnormally high stress response to cold sensitization and/or exposure in the subject before administration of the dual N-type and L-type calcium channel blocker selective for the N-type calcium channel. In some embodiments, the method further comprises determining a reduction in stress response to cold sensitization and/or exposure in the subject after administration of the dual N-type and L-type calcium channel blocker selective for the N-type calcium channel.

In another aspect, disclosed herein is a method of treating a disease or disorder associated with dysregulation of blood flow and sympathetic nervous system overactivity in a subject in need thereof, comprising: (1) determining that the subject has TRP-v1 overactivation; and (2) administering a therapeutically effective amount of a dual N-type and L-type calcium channel blocker selective for the N-type calcium channel to the subject.

In some aspects, disclosed herein is a method of treating a disease or disorder associated with dysregulation of blood flow and sympathetic nervous system overactivity in a subject showing a clinical record of TRP-v1 overactivation in need thereof, comprising administering a therapeutically effective amount of a dual N-type and L-type calcium channel blocker selective for the N-type calcium channel to the subject.

In some aspects, disclosed herein is a method of treating a disease or disorder associated with dysregulation of blood flow and sympathetic nervous system overactivity in a subject in need thereof, comprising (1) administering a therapeutically effective amount of a dual N-type and L-type calcium channel blocker selective for the N-type calcium channel to the subject; and (2) determining a reduction of TRP-v1 activity in the subject.

In some embodiments, the disease or disorder associated with dysregulation of blood flow and sympathetic nervous system overactivity is a disease or disorder characterized by vasoconstriction or neuropathic pain.

In some embodiments, the disease or disorder associated with dysregulation of blood flow and sympathetic nervous system overactivity is characterized by neuropathic pain (e.g., chronic neuropathic pain), vasoconstriction, dysesthetic pain, burning pain, hyperesthetic pain, allodynia, lancinating pain, crampy pain, dull pain, body temperature changes of the subject, hyperesthesia, changes in skin or tissue color, edema, changes in skin turgor, rubor, pallor, cyanosis, vasospasm, or any combination thereof. In some embodiments, the disease or disorder is selected from the group consisting of: Raynaud's syndrome (e.g., primary Raynaud's syndrome or secondary Raynaud's syndrome); scleroderma or systemic sclerosis; complex regional pain syndrome Type I; complex regional pain syndrome Type II; nerve pain after surgery; burning pain during or after nerve compression; perception of temperature changes during or after nerve compression; pain during or after a burn; burning dysesthesias; neuropathic pain; erythromelalgia; vascular mediated pain syndromes; spinal stenosis; lumbar radiculopathy; failed back syndrome; cervical radiculopathy; causalgia; sympathetically mediated pain syndromes; trigeminal neuralgia; post-herpetic neuralgia; causalgia; fibromyalgia; diabetic neuropathy; chemotherapy induced neuropathy; restless legs syndrome; hot flashes; atherosclerosis, kidney disease or dysfunction, post-operative renal dysfunction, arthritis-related pain (e.g., osteoarthritis-related pain), drug related neuropathic pain, diseases of endothelial dysfunction, cardiac left ventricular disease or dysfunction; limb, extremity, surgical flap, post-surgical ischemia, or acute limb or extremity ischemia as a consequence of vasospasm or a thrombotic event; osteoporosis; heart remodeling after atrial fibrillation, QT prolongation in patients at risk for cardiovascular disease including hemodialysis patients; postoperative pain; hypertension; or treatment-resistant hypertension wherein other antihypertensive medications including but not limited to ace inhibitors, angiotensin receptor blockade agents, beta blockers, diuretics, alpha blockers, and other calcium channel blockers have dose limitations due to efficacy limitations or side effect occurrence. In certain of these embodiments, the disease or disorder is Raynaud's syndrome. For example, the Raynaud's syndrome is selected from the group consisting of: primary Raynaud's syndrome; secondary Raynaud's syndrome; Raynaud's syndrome of the nipple, nose, ear, penis, tongue, and/or any alar circulatory region. For example, the Raynaud's syndrome is secondary Raynaud's syndrome.

In some aspects, disclosed herein is a method of treating a disease or disorder characterized by vasoconstriction or neuropathic pain in a subject in need thereof, the method comprising (a) determining that the disease or disorder is associated with vasoconstriction or neuropathic pain; (b) determining that the subject has TRP-v1 overactivity; and (c) administering to the subject a therapeutically effective amount of a dual N-type and L-type calcium channel blocker selective for the N-type calcium channel.

In some aspects, disclosed herein is a method of treating a disease or disorder characterized by vasoconstriction or neuropathic pain in a subject showing a clinical record of TRP-v1 overactivation in need thereof, the method comprising (a) determining that the disease or disorder is associated with vasoconstriction or neuropathic pain; and (b) administering to the subject a therapeutically effective amount of a dual N-type and L-type calcium channel blocker selective for the N-type calcium channel.

In some aspects, disclosed herein is a method of treating a disease or disorder characterized by vasoconstriction or neuropathic pain in a subject in need thereof, the method comprising (a) determining that the disease or disorder is associated with vasoconstriction or neuropathic pain; (b) administering to the subject a therapeutically effective amount of a dual N-type and L-type calcium channel blocker selective for the N-type calcium channel; and (c) determining a reduction of TRP-v1 activity in the subject.

In some embodiments, the disease or disorder characterized by vasoconstriction or neuropathic pain is characterized by neuropathic pain (e.g., chronic neuropathic pain), vasoconstriction, dysesthetic pain, hyperesthetic pain, allodynia, lancinating pain, crampy pain, dull pain, burning pain, body temperature changes of the subject, hyperesthesia, changes in skin or tissue color, edema, changes in skin turgor, rubor, pallor, cyanosis, vasospasm, or any combination thereof. In some embodiments, the disease or disorder is selected from the group consisting of: Raynaud's syndrome (e.g., primary Raynaud's syndrome or secondary Raynaud's syndrome); scleroderma or systemic sclerosis; complex regional pain syndrome Type I; complex regional pain syndrome Type II; nerve pain after surgery; burning pain during or after nerve compression; perception of temperature changes during or after nerve compression; pain during or after a burn; burning dysesthesias; neuropathic pain; erythromelalgia; vascular mediated pain syndromes; spinal stenosis; lumbar radiculopathy; failed back syndrome; cervical radiculopathy; causalgia; sympathetically mediated pain syndromes; trigeminal neuralgia; post-herpetic neuralgia; causalgia; fibromyalgia; diabetic neuropathy; chemotherapy induced neuropathy; restless legs syndrome; hot flashes; atherosclerosis, kidney disease or dysfunction, post-operative renal dysfunction, arthritis-related pain (e.g., osteoarthritis-related pain), drug related neuropathic pain, diseases of endothelial dysfunction, cardiac left ventricular disease or dysfunction; limb, extremity, surgical flap, post-surgical ischemia, or acute limb or extremity ischemia as a consequence of vasospasm or a thrombotic event; osteoporosis; heart remodeling after atrial fibrillation, QT prolongation in patients at risk for cardiovascular disease including hemodialysis patients; postoperative pain; hypertension; or treatment-resistant hypertension wherein other antihypertensive medications including but not limited to ace inhibitors, angiotensin receptor blockade agents, beta blockers, diuretics, alpha blockers, and other calcium channel blockers have dose limitations due to efficacy limitations or side effect occurrence. In certain of these embodiments, the disease or disorder is Raynaud's syndrome. For example, the Raynaud's syndrome is selected from the group consisting of: primary Raynaud's syndrome; secondary Raynaud's syndrome; Raynaud's syndrome of the nipple, nose, ear, penis, tongue, and/or any alar circulatory region. For example, the Raynaud's syndrome is secondary Raynaud's syndrome.

Additional methods and compositions for treating various diseases and disorders with a dual N-type and L-type calcium channel blocker selective for the N-type calcium channel can be found in WO2021/178903 and PCT/US2021/060778, each of which is incorporated by reference herein in its entirety.

### Pharmaceutical Compositions and Formulations

In some aspects, disclosed herein is a pharmaceutical composition comprising a dual N-type and L-type calcium channel blocker selective for the N-type calcium channel, an agent that increases blood pressure, and optionally a pharmaceutically acceptable excipient.

In some aspects, disclosed herein is a pharmaceutical composition comprising a dual N-type and L-type calcium channel blocker selective for the N-type calcium channel; a therapeutic agent selected from the group consisting of: riociguat, amlodipine, pregabalin, gabapentin, nifedipine, nicardipine, conotoxins, cadmium, caroverine, levetiracetam, lamotrigine, NP078585, pregabalin, TROX-1, non-steroidal anti-inflammatory agents (e.g., ibuprofen), valsartan, dimethylsulfoxide, N-acetylcysteine, N-acetyl cysteine, 4-hydroxy-2,2,6,6-tetramethylpiperidin-1-oxyl, magnesium sulfate, ziconotide, analgesics (e.g., acetaminophen), anesthetics, anticonvulsants, antidepressants, oral muscle relaxants, corticosteroids, calcitonin, bisphosphonates (e.g., neridronate), cyclooxygenase (COX)-2 inhibitors, free-radical scavenger agents (e.g., ketoprofen), prednisolone, prednisone, oral steroids, opioids, riociguat, amlodipine, pregabalin, alendronate, pamidronate, gabapentin, nifedipine, nicardipine, bupivacaine, epinephrine, caroverine, ascorbic acid (Vitamin C), valsartan, dimethylsulfoxide, N-acetylcysteine, phenoxybenzamine, or a combination thereof; and optionally a pharmaceutically acceptable excipient.

In some aspects, disclosed herein is a pharmaceutical composition comprising a dual N-type and L-type calcium channel blocker selective for the N-type calcium channel; a therapeutic agent selected from the group consisting of: oxycodone, tramadol, Dilaudid, OxyContin, Cymbalta, a statin, gabapentin, pregabalin, an angiotensin-converting-enzyme (ACE) inhibitor, an angiotensin receptor blocker (ARB), niacin, a proton pump inhibitor, aspirin, Fentanyl Transdermal System, acetaminophen/oxycodone, Roxicodone, Ultram, hydromorphone, Percocet, MS Contin, Butrans, morphine, hydromorphone, methadone, buprenorphine, duloxetine, fentanyl, Duragesic, Endocet, Roxanol, Kadian, Roxicet, ConZip, Methadose, Oxyfast, Dazidox, Fentora, Irenka, Methadone Diskets, Oramorph SR, Roxicodone Intensol, Xtampza ER, Actiq, Belbuca, ETH-Oxydose, Infumorph, naloxone/pentazocine, Oxaydo, Oxydose, OxyIR, ziconotide, Abstral, Astramorph PF, Buprenex, Dolophine, Duramorph, Duramorph PF, Embeda, Lazanda, MorphaBond ER, morphine/naltrexone, Prialt, RMS, Roxanol-T, Sublimaze, Subsys, Talwin Nx, Magnacet, Nalocet, Narvox, Perloxx, Primlev, Xolox, and Prolate; and optionally a pharmaceutically acceptable excipient.

In some aspects, disclosed herein is a pharmaceutical composition comprising a dual N-type and L-type calcium channel blocker selective for the N-type calcium channel; a therapeutic agent selected from the group consisting of: an aluminum antacid, a magnesium antacid, a calcium channel blocker that is different from the dual N-type and L-type calcium channel blocker selective for the N-type calcium channel (e.g., a non-N-selective calcium channel blocker (e.g., diltiazem, amlodipine, or verapamil)), colchicine, warfarin, prednisone, a diphosphonate, and sodium etidronate; and optionally a pharmaceutically acceptable excipient.

In some aspects, disclosed herein is a pharmaceutical composition comprising a dual N-type and L-type calcium channel blocker selective for the N-type calcium channel; an opioid; and optionally a pharmaceutically acceptable excipient.

In some embodiments, the opioid is selected from the group consisting of: fentanyl, heroin, hydromorphone, oxymorphone, methadone, oxycodone, morphine, hydrocodone, codeine, meperidine, tramadol, and combinations thereof. In some embodiments, the opioid is oxycodone. In some embodiments, the opioid is hydromorphone. In some embodiments, the opioid is oxymorphone. In some embodiments, the opioid is methadone. In some embodiments, the opioid is hydrocodone. In some embodiments, the opioid is meperidine. In some embodiments, the opioid is tramadol.

In some embodiments, the amount of the opioid (e.g., oxycodone) is from about 1 μg to about 100 mg (e.g., from about 1 μg to about 5 mg, from about 1 mg to about 50 mg, from about 1 mg to about 25 mg, from about 1 mg to about 15 mg, from about 1 mg to about 10 mg, from about 1 mg to about 5 mg, from about 1 mg to about 3 mg, from about 3 mg to about 35 mg, from about 5 mg to about 25 mg, from about 8 mg to about 28 mg, from about 12 mg to about 28 mg, from about 9 mg to about 21 mg, from about 15 mg to about 25 mg, from about 20 mg to about 30 mg, from about 22 mg to about 28 mg, from about 17 mg to about 23 mg, from about 8 mg to about 12 mg, about 8 mg, about 0.1 mg, about 0.5 mg, about 1 mg, about 2 mg, about 3 mg, about 4 mg, about 5 mg, about 6 mg, about 8 mg, about 10 mg, about 12 mg, about 18 mg, about 20 mg, about 22 mg, or about 25 mg). In some embodiments, the amount of the opioid is from about 1 mg to about 25 mg. In some embodiments, the amount of the opioid is from about 1 mg to about 15 mg. In some embodiments, the amount of the opioid is about 1 mg. In some embodiments, the amount of the opioid is about 3 mg. In some embodiments, the amount of the opioid is about 5 mg. In some embodiments, the amount of the opioid is about 10 mg. In some embodiments, the amount of the opioid is about 20 mg. In some embodiments, the amount of the opioid is about 25 mg.

In some aspects, disclosed herein is a pharmaceutical composition comprising a dual N-type and L-type calcium channel blocker selective for the N-type calcium channel; oxycodone; and optionally a pharmaceutically acceptable excipient.

In some aspects, disclosed herein is a pharmaceutical composition comprising cildinipine; oxycodone; and optionally a pharmaceutically acceptable excipient.

In some embodiments, the dual N-type and L-type calcium channel blocker selective for the N-type calcium channel is selected from the group consisting of: cilnidipine, Z-160, zicinotide, ralfinamide, CNV2197944, or pharmaceutically acceptable salts thereof.

In some embodiments, the dual N-type and L-type calcium channel blocker selective for the N-type calcium channel is selected from the group consisting of: cilnidipine, Z-160, ralfinamide, CNV2197944, or pharmaceutically acceptable salts thereof.

In some embodiments, the dual N-type and L-type calcium channel blocker selective for the N-type calcium channel is selected from the group consisting of: cilnidipine, Z-160, CNV2197944, or pharmaceutically acceptable salts thereof.

In some embodiments, the dual N-type and L-type calcium channel blocker selective for the N-type calcium channel is selected from the group consisting of: cilnidipine, Z-160, zicinotide, or pharmaceutically acceptable salts thereof.

In some embodiments, the dual N-type and L-type calcium channel blocker selective for the N-type calcium channel is cilnidipine or a pharmaceutically acceptable salt thereof.

In some embodiments, the dual N-type and L-type calcium channel blocker selective for the N-type calcium channel is CNV2197944.

In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines.

In some aspects, disclosed herein is a pharmaceutical composition comprising a dual N-type and L-type calcium channel blocker selective for the N-type calcium channel, an agent that treats erectile dysfunction, and optionally a pharmaceutically acceptable excipient.

In some embodiments, the dual N-type and L-type calcium channel blocker selective for the N-type calcium channel is selected from the group consisting of: cilnidipine, Z-160, zicinotide, ralfinamide, CNV2197944, or pharmaceutically acceptable salts thereof.

In some embodiments, the dual N-type and L-type calcium channel blocker selective for the N-type calcium channel is selected from the group consisting of: cilnidipine, Z-160, ralfinamide, CNV2197944, or pharmaceutically acceptable salts thereof.

In some embodiments, the dual N-type and L-type calcium channel blocker selective for the N-type calcium channel is selected from the group consisting of: cilnidipine, Z-160, CNV2197944, or pharmaceutically acceptable salts thereof.

In some embodiments, the dual N-type and L-type calcium channel blocker selective for the N-type calcium channel is selected from the group consisting of: cilnidipine, Z-160, zicinotide, or pharmaceutically acceptable salts thereof.

In some embodiments, the dual N-type and L-type calcium channel blocker selective for the N-type calcium channel is cilnidipine or a pharmaceutically acceptable salt thereof.

In some embodiments, the dual N-type and L-type calcium channel blocker selective for the N-type calcium channel is CNV2197944.

In some embodiments, the agent that treats erectile dysfunction is sildenafil, tadalafil, or phosphodiesterase type 5 inhibitors.

In some aspects, disclosed herein is a pharmaceutical composition comprising a dual N-type and L-type calcium channel blocker selective for the N-type calcium channel; cyclophosphamide, nintedanib, methotrexate, mycophenolate, a glucocorticoid (e.g., prednisone, dexamethasone, and hydrocortisone), a non steroidal anti-inflammatory drug (e.g., aspirin, ibuprofen, or naproxen), D-penicillamine, a diuretic, omeprazole, bosentan, epoprostenol, enalapril, lisinopril, captopril, or any combination thereof; and optionally a pharmaceutically acceptable excipient.

In some aspects, disclosed herein is a pharmaceutical composition comprising a dual N-type and L-type calcium channel blocker selective for the N-type calcium channel; riociguat, amlodipine, pregabalin, alendronate, prednisone, pamidronate, gabapentin, nifedipine, nicardipine, conotoxins, cadmium, bupivacaine, epinephrine, caroverine, vitamin C, N-acetyl cysteine, 4-hydroxy-2,2,6,6-tetramethylpiperidin-1-oxyl, magnesium sulfate, levetiracetam, lamotrigine, NP078585, pregabalin, TROX-1, acetaminophen, non-steroidal anti-inflammatory agents (e.g., ibuprofen), valsartan, dimethylsulfoxide, N-acetylcysteine, ziconotide, or any combination thereof; and optionally a pharmaceutically acceptable excipient.

In some aspects, disclosed herein is a pharmaceutical composition comprising a dual N-type and L-type calcium channel blocker selective for the N-type calcium channel; nintedanib; and optionally a pharmaceutically acceptable excipient.

In some aspects, disclosed herein is a pharmaceutical composition comprising a dual N-type and L-type calcium channel blocker selective for the N-type calcium channel; an antimalarial drug (e.g., hydroxychloroquine), a non-steroidal anti-inflammatory drug (e.g., aspirin, ibuprofen, or naproxen), belimumab, a corticosteroid (e.g., prednisone or prednisolone), an immunosuppressant (e.g., azathioprine, cyclophosphamide, methotrexate, and mycophenolate mofetil), or any combination thereof; and optionally a pharmaceutically acceptable excipient.

In some aspects, disclosed herein is a pharmaceutical composition comprising a dual N-type and L-type calcium channel blocker selective for the N-type calcium channel; disease-modifying anti-rheumatic drugs (e.g., methotrexate or sulfasalazine), a non-steroidal anti-inflammatory drug (e.g., aspirin, ibuprofen, or naproxen), a corticosteroid (e.g., prednisone or prednisolone), a biologic (e.g., anakinra or tocilizumab), or any combination thereof; and optionally a pharmaceutically acceptable excipient.

In some aspects, disclosed herein is a pharmaceutical composition comprising a dual N-type and L-type calcium channel blocker selective for the N-type calcium channel; plaquenil, an antimalarial drug (e.g., hydroxychloroquine), evoxac, cevimeline, infliximab, or any combination thereof; and optionally a pharmaceutically acceptable excipient.

In some aspects, disclosed herein is a pharmaceutical composition comprising a dual N-type and L-type calcium channel blocker selective for the N-type calcium channel; nintedanib, pirfenidone, or any combination thereof; and optionally a pharmaceutically acceptable excipient.

In some aspects, disclosed herein is a pharmaceutical composition comprising a dual N-type and L-type calcium channel blocker selective for the N-type calcium channel, a calcineurin inhibitor, and optionally a pharmaceutically acceptable excipient.

In some embodiments, the calcineurin inhibitor is a cyclosporine.

In some embodiments, the pharmaceutical composition further comprises a non-steroidal anti-inflammatory drug. In some embodiments, the non-steroidal anti-inflammatory drug is aspirin.

In some embodiments, the dual N-type and L-type calcium channel blocker selective for the N-type calcium channel is selected from the group consisting of: cilnidipine, Z-160, zicinotide, ralfinamide, CNV2197944, or pharmaceutically acceptable salts thereof. In some embodiments, the dual N-type and L-type calcium channel blocker selective for the N-type calcium channel is selected from the group consisting of: cilnidipine, Z-160, ralfinamide, CNV2197944, or pharmaceutically acceptable salts thereof. In some embodiments, the dual N-type and L-type calcium channel blocker selective for the N-type calcium channel is selected from the group consisting of: cilnidipine, Z-160, CNV2197944, or pharmaceutically acceptable salts thereof. In some embodiments, the dual N-type and L-type calcium channel blocker selective for the N-type calcium channel is selected from the group consisting of: cilnidipine, Z-160, or pharmaceutically acceptable salts thereof. In some embodiments, the dual N-type and

L-type calcium channel blocker selective for the N-type calcium channel is a dihydropyridine N-type calcium channel blocker. In certain of these embodiments, the dual N-type and L-type calcium channel blocker selective for the N-type calcium channel is cilnidipine or a pharmaceutically acceptable salt thereof. In some embodiments, the dual N-type and L-type calcium channel blocker selective for the N-type calcium channel is CNV2197944.

In some embodiments, the dual N-type and L-type calcium channel blocker selective for the N-type calcium channel and/or sodium channel blocker can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, oral, parenteral, transdermal, intranasal, sublingual, neuraxial, or ocular.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In some embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In some embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosing and Regimens

In some embodiments, the dosage of the dual N-type and L-type selective calcium blocker (e.g., cilnidipine) is from about 1 mg to about 100 mg (e.g., from about 1 mg to about 50 mg, from about 1 mg to about 25 mg, from about 1 mg to about 15 mg, from about 1 mg to about 10 mg, from about 1 mg to about 5 mg, from about 1 mg to about 3 mg, from about 3 mg to about 35 mg, from about 5 mg to about 25 mg, from about 8 mg to about 28 mg, from about 12 mg to about 28 mg, from about 9 mg to about 21 mg, from about 15 mg to about 25 mg, from about 20 mg to about 30 mg, from about 22 mg to about 28 mg, from about 17 mg to about 23 mg, from about 8 mg to about 12 mg, about 8 mg, about 2 mg, about 4 mg, about 6 mg, about 8 mg, about 10 mg, about 12 mg, about 18 mg, about 20 mg, about 22 mg, or about 25 mg). In some embodiments, the dosage of the dual N-type and L-type selective calcium blocker is from about 5 mg to about 25 mg. In some embodiments, the dosage of the dual N-type and L-type selective calcium blocker is from about 9 mg to about 21 mg. In some embodiments, the dosage of the dual N-type and L-type selective calcium blocker is about 10 mg. In some embodiments, the dosage of the dual N-type and L-type selective calcium blocker is about 20 mg. In some embodiments, the dosage of the dual N-type and L-type selective calcium blocker is about 25 mg.

The foregoing dosages can be administered on a daily basis (e.g., as a single dose or as two or more (e.g., 2, 3, 4, or 5) divided doses) or non-daily basis (e.g., every other day, every two days, every three days, once weekly, twice weeks, once every two weeks, once a month).

In some embodiments of the methods disclosed herein, the dual N-type and L-type calcium channel blocker selective for the N-type calcium channel is administered orally, parenterally, transdermally, intranasally, sublingually, neuraxially, or ocularly. In some embodiments, the dual N-type and L-type calcium channel blocker selective for the N-type calcium channel is administered orally.

In some embodiments, the dual N-type and L-type calcium channel blocker selective for the N-type calcium channel and the at least one additional therapeutic agent and/or the phosphodiesterase type 5 inhibitor are administered separately, sequentially, or simultaneously. In some embodiments, the dual N-type and L-type calcium channel blocker selective for the N-type calcium channel and the at least one additional therapeutic agent and/or the phosphodiesterase type 5 inhibitor are administered separately. In some embodiments, the dual N-type and L-type calcium channel blocker selective for the N-type calcium channel and the at least one additional therapeutic agent and/or the phosphodiesterase type 5 inhibitor are administered sequentially. In some embodiments, the dual N-type and L-type calcium channel blocker selective for the N-type calcium channel and the at least one additional therapeutic agent and/or the phosphodiesterase type 5 inhibitor are administered simultaneously. In some embodiments, the du dual N-type and L-type calcium channel blocker selective for the N-type calcium channel and the at least one additional therapeutic agent and/or the phosphodiesterase type 5 inhibitor are administered simultaneously as a fixed dosage form.

In some embodiments, each administration of the dual N-type and L-type selective calcium blocker and the at least one additional therapeutic agent and/or the phosphodiesterase type 5 inhibitor is less frequent than the frequency of administering the dual N-type and L-type calcium channel blocker selective for the N-type calcium channel alone useful to treat the calcinosis.

In some embodiments, each administration of the dual N-type and L-type calcium channel blocker selective for the N-type calcium channel is separated by at least about 1 hour (e.g., about 1 to about 48 hours, about 1 to about 12 hours, about 12 to about 24 hours, about 2 to about 10 hours, about 4 to about 12 hours, about 6 to about 10 hours, about 10 to about 16 hours, about 14 to about 22 hours, about 16 to about 24 hours, about 24 to about 30 hours, about 30 to about 36 hours, about 36 to about 42 hours, about 40 to about 48 hours, at least about 2 hours, at least about 4 hours, at least about 6 hours, at least about 8 hours, at least about 12 hours, at least about 15 hours, at least about 20 hours, at least about 24 hours, at least about 36 hours, at least about 48 hours, at least about 72 hours, at least about 4 days, at least about 5 days, at least about 3 days, at least about 5 days, at least about 1 week). In some embodiments, each administration of the dual N-type and L-type calcium channel blocker selective for the N-type calcium channel is separated by at least about 24 hours. In some embodiments, each administration of the dual N-type and L-type calcium channel blocker selective for the N-type calcium channel is separated by at least about 48 hours. In some embodiments, each administration of the dual N-type and L-type calcium channel blocker selective for the N-type calcium channel is separated by at least about 72 hours. In some embodiments, each administration of the dual N-type and L-type calcium channel blocker selective for the N-type calcium channel is separated by at least about 1 week. In some embodiments, each administration of the dual N-type and L-type calcium channel blocker selective for the N-type calcium channel is separated by about 4 hours. In some embodiments, each administration of the dual N-type and L-type calcium channel blocker selective for the N-type calcium channel is separated by about 4 to about 12 hours. In some embodiments, each administration of the dual N-type and L-type calcium channel blocker selective for the N-type calcium channel is separated by at least about 1 week.

In some embodiments, after administration of the dual N-type and L-type calcium channel blocker selective for the N-type calcium channel, the subject experiences gastrointestinal symptoms that are ameliorated by the consumption of food prior to administration of the dual N-type and L-type calcium channel blocker selective for the N-type calcium channel. In some embodiments, the subject consumes food up to about 6 hours before administering the dual N-type and L-type calcium channel blocker selective for the N-type calcium channel. For example, the subject consumes food up to about 5 hours, about 4 hours, about 3 hours, about 2 hours, about 1 hour, about 30 minutes, about 20 minutes, about 15 minutes, about 10 minutes, about 5 minutes, about 1 minute, about 30 seconds, or about 5 seconds before administering the dual N-type and L-type calcium channel blocker selective for the N-type calcium channel. For example, the subject consumes food concurrently with administering the dual N-type and L-type calcium channel blocker selective for the N-type calcium channel.

In some embodiments, the period of administration of a the dual N-type and L-type calcium channel blocker selective for the N-type calcium channel is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, about 1 day to about 1 month, about 1 day, to about two weeks, at least about 1 month, 4 months, 5 months, 6 months, 7 months, 8 months, 9 months, 10 months, 11 months, 12 months, at least about 1 year, or more. In some embodiments, the period of administration of the dual N-type and L-type calcium channel blocker selective for the N-type calcium channel is for at least about one month. In some embodiments, the period of administration of the dual N-type and L-type calcium channel blocker selective for the N-type calcium channel is for at least about one year. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, the dual N-type and L-type calcium channel blocker selective for the N-type calcium channel is administered to an individual for a period of time followed by a separate period of time. In another embodiment, the dual N-type and L-type calcium channel blocker selective for the N-type calcium channel is administered for a first period and a second period following the first period, with administration stopped during the second period, followed by a third period where administration of the dual N-type and L-type calcium channel blocker selective for the N-type calcium channel is started and then a fourth period following the third period where administration is stopped. In an aspect of this embodiment, the period of administration of the dual N-type and L-type calcium channel blocker selective for the N-type calcium channel followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, the dual N-type and L-type selective calcium blocker is formulated to maintain the plasma level of the dual N-type and L-type selective calcium blocker in the subject at 10% or greater (e.g., 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, or 95% or greater) of the peak dual N-type and L-type selective calcium blocker plasma level for at least 6 hours (e.g., at least 8 hours, at least 12 hours, at least 16 hours, at least 20 hours, at least 24 hours, at least 36 hours, or at least 48 hours) after administration of the dual N-type and L-type selective calcium blocker. It is understood that the peak dual N-type and L-type selective calcium blocker plasma level is the highest plasma concentration of the dual N-type and L-type selective calcium blocker observed in the subject after administration of the dual N-type and L-type selective calcium blocker.

### Exemplary Embodiments

Embodiment 1. A method of treating complex regional pain syndrome in a subject in need thereof, comprising administering to the subject a therapeutically effective amount of a dual N-type and L-type calcium channel blocker selective for the N-type calcium channel.

Embodiment 2. A method of treating pain in a subject in need thereof, comprising administering to the subject a therapeutically effective amount of a dual N-type and L-type calcium channel blocker selective for the N-type calcium channel; wherein the subject has complex regional pain syndrome.

Embodiment 3. The method of any one of embodiments 1-2, wherein the complex regional pain syndrome is identified or diagnosed in a leg of the subject.

Embodiment 4. The method of any one of embodiments 1-2, wherein the complex regional pain syndrome is identified or diagnosed in an arm of the subject.

Embodiment 5. The method of any one of embodiments 1-2, wherein the complex regional pain syndrome is associated with an injury in the subject.

Embodiment 6. The method of embodiment 5, wherein the injury is a bone fracture or a crushing injury to a nerve.

Embodiment 7. The method of any one of embodiments 1-2, wherein the complex regional pain syndrome is associated with a stroke in the subject.

Embodiment 8. The method of any one of embodiments 1-2, wherein the complex regional pain syndrome is associated with a heart attack in the subject.

Embodiment 9. The method of any one of embodiments 1-8, wherein the complex regional pain syndrome is complex regional pain syndrome Type I.

Embodiment 10. The method of any one of embodiments 1-8, wherein the complex regional pain syndrome is complex regional pain syndrome Type II.

Embodiment 11. The method of any one of embodiments 1-10, wherein after administration of the therapeutically effective amount of the dual N-type and L-type calcium channel blocker selective for the N-type calcium channel, the severity, frequency, and/or duration of burning or throbbing pain, sensitivity to cold, swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, or any combination thereof are reduced in the subject.

Embodiment 12. The method of embodiment 2, wherein the pain is neuropathic pain.

Embodiment 13. The method of any one of embodiments 1-12, wherein the dual N-type and L-type calcium channel blocker selective for the N-type calcium channel exhibits at least a 50-fold selectivity for the N-type calcium channel over an L-type calcium channel.

Embodiment 14. The method of any one of embodiments 1-13, wherein the dual N-type and L-type calcium channel blocker selective for the N-type calcium channel exhibits a 50-fold to 100-fold selectivity for the N-type calcium channel over an L-type calcium channel.

Embodiment 15. The method of any one of embodiments 1-14, wherein the dual N-type and L-type calcium channel blocker selective for the N-type calcium channel is selected from the group consisting of: cilnidipine, Z-160, CNV2197944, or pharmaceutically acceptable salts thereof.

Embodiment 16. The method of any one of embodiments 1-15, wherein the dual N-type and L-type calcium channel blocker selective for the N-type calcium channel is cilnidipine or a pharmaceutically acceptable salt thereof.

Embodiment 17. The method of embodiment 16, wherein the dosage of the cilnidipine is from about 5 mg to about 25 mg.

Embodiment 18. The method of embodiment 16, wherein the dosage of the cilnidipine is from about 9 mg to about 21 mg.

Embodiment 19. The method of embodiment 16, wherein the dosage of the cilnidipine is about 10 mg.

Embodiment 20. The method of embodiment 16, wherein the dosage of the cilnidipine is about 20 mg.

Embodiment 21. The method of any one of embodiments 1-20, wherein the dual N-type and L-type calcium channel blocker selective for the N-type calcium channel is administered orally.

Embodiment 22. The method of any one of embodiments 1-21, wherein each administration of the dual N-type and L-type calcium channel blocker selective for the N-type calcium channel is separated by at least about 8 hours.

Embodiment 23. The method of any one of embodiments 1-22, wherein each administration of the dual N-type and L-type calcium channel blocker selective for the N-type calcium channel is separated by at least about 24 hours.

Embodiment 24. The method of any one of embodiments 1-23, wherein each administration of the dual N-type and L-type calcium channel blocker selective for the N-type calcium channel is separated by at least about 48 hours.

Embodiment 25. The method of any one of embodiments 1-24, wherein each administration of the dual N-type and L-type calcium channel blocker selective for the N-type calcium channel is separated by at least about 1 week.

Embodiment 26. The method of any one of embodiments 1-25, wherein the period of administration of the dual N-type and L-type calcium channel blocker selective for the N-type calcium channel is from about 1 day to about 1 month.

Embodiment 27. The method of any one of embodiments 1-25, wherein the period of administration of the dual N-type and L-type calcium channel blocker selective for the N-type calcium channel is from about 1 day to about two weeks.

Embodiment 28. The method of any one of embodiments 1-25, wherein the period of administration of the dual N-type and L-type calcium channel blocker selective for the N-type calcium channel is about two weeks.

Embodiment 29. The method of any one of embodiments 1-25, wherein the period of administration of the dual N-type and L-type calcium channel blocker selective for the N-type calcium channel is about 12 days.

Embodiment 30. The method of any one of embodiments 1-25, wherein the period of administration of the dual N-type and L-type calcium channel blocker selective for the N-type calcium channel is about one week.

Embodiment 31. The method of any one of embodiments 1-30, wherein the dual N-type and L-type calcium channel blocker selective for the N-type calcium channel further inhibits a Nav 1.7 sodium channel.

Embodiment 32. The method of any one of embodiments 1-31, wherein the dual N-type and L-type calcium channel blocker selective for the N-type calcium channel further inhibits a TRPv-1 channel.

Embodiment 33. The method of any one of embodiments 1-32, further comprising determining that the subject has TRPv-1 overactivation.

Embodiment 34. The method of embodiment 33, wherein determining that the subject has TRPv-1 overactivation comprises determining an abnormally high TRP-v1 current density.

Embodiment 35. The method of any one of embodiments 1-34, wherein the subject has a clinical record showing TRPv-1 overactivation in the subject.

Embodiment 36. The method of any one of embodiments 1-35, further comprising determining a decrease in TRPv-1 activation or upregulation in the subject after administration of the dual N-type and L-type calcium channel blocker selective for the N-type calcium channel.

Embodiment 37. The method of any one of embodiments 1-36, further comprising determining an abnormally high inflammation in the subject before administration of the dual N-type and L-type calcium channel blocker selective for the N-type calcium channel.

Embodiment 38. The method of any one of embodiments 1-37, further comprising determining a reduction in inflammation in the subject after administration of the dual N-type and L-type calcium channel blocker selective for the N-type calcium channel.

Embodiment 39. The method of any one of embodiments 37-38, wherein the inflammation is generalized, neurogenic, or both.

Embodiment 40. The method of any one of embodiments 1-39, further comprising determining that the subject has abnormally elevated concentrations of TNF-α, IL-1B. IL2, IL-6, ET-I, cGRP, bradykinin, substance P, MMP9, CXCL13, or any combination thereof before administration of the dual N-type and L-type calcium channel blocker selective for the N-type calcium channel.

Embodiment 41. The method of any one of embodiments 1-40, further comprising determining a lower concentration of TNF-α, IL-1B. IL2, IL-6, ET-I, cGRP, bradykinin, substance P, MMP9, CXCL13, or any combination thereof in the subject after administration of the dual N-type and L-type calcium channel blocker selective for the N-type calcium channel.

Embodiment 42. The method of any one of embodiments 40-41, wherein the abnormally elevated concentrations of TNF-α, IL-1B. IL2, IL-6, ET-I, cGRP, bradykinin, substance P, MMP9, CXCL13, or any combination thereof are measured in the cerebrospinal fluid or the plasma of the subject.

Embodiment 43. The method of any one of embodiments 1-42, further comprising determining that the subject has an abnormally high immunologic response, an abnormally high number or concentration of non-specific autoantibodies to sympathetic receptors, or both before administration of the dual N-type and L-type calcium channel blocker selective for the N-type calcium channel.

Embodiment 44. The method of any one of embodiments 1-43, further comprising determining a reduction in immunologic response, a reduction in number or concentration of non-specific autoantibodies to sympathetic receptors, or both after administration of the dual N-type and L-type calcium channel blocker selective for the N-type calcium channel.

Embodiment 45. The method of any one of embodiments 1-44, further comprising determining autonomic dysregulation; an abnormally high number of sympathetic receptors; an abnormally high concentration of circulating catecholamines; sympathetic hyperactivity; or any combination thereof in the subject before administration of the dual N-type and L-type calcium channel blocker selective for the N-type calcium channel.

Embodiment 46. The method of any one of embodiments 1-45, further comprising determining an improvement in autonomic functioning; a reduction in sympathetic receptors; a reduction in the concentration of circulating catecholamines; a reduction in sympathetic activity; or any combination thereof in the subject after administration of the dual N-type and L-type calcium channel blocker selective for the N-type calcium channel.

Embodiment 47. The method of any one of embodiments 1-46, further comprising determining an abnormally high central sensitization and neuroplasticity with increased substance P, bradykinin, and/or glutamate; an overexpression of spinal NMDA receptors; gabaminergic overactivity; or any combination thereof in the subject before administration of the dual N-type and L-type calcium channel blocker selective for the N-type calcium channel.

Embodiment 48. The method of any one of embodiments 1-47, further comprising determining a reduction in central sensitization and neuroplasticity with increased substance P, bradykinin, and/or glutamate; an overexpression of spinal NMDA receptors; gabaminergic overactivity; or any combination thereof in the subject after administration of the dual N-type and L-type calcium channel blocker selective for the N-type calcium channel.

Embodiment 49. The method of any one of embodiments 1-48, further comprising determining peripheral oversensitization; an abnormally high concentration of spinal N-methyl-D-aspartate; abnormally high concentration of spinal glutamate; upregulation in TNF-α and/or TRP-V1 activation; or any combination thereof in the subject before administration of the dual N-type and L-type calcium channel blocker selective for the N-type calcium channel.

Embodiment 50. The method of any one of embodiments 1-49, further comprising determining a reduction in peripheral sensitization; a reduction in spinal N-methyl-D-aspartate; a reduction in spinal glutamate; a reduction in upregulation of TNF-α and/or TRP-V1 activation; or any combination thereof in the subject after administration of the dual N-type and L-type calcium channel blocker selective for the N-type calcium channel.

Embodiment 51. The method of any one of embodiments 1-50, further comprising determining abnormally high oxidative stress, upregulation of superoxide dismutase, upregulation of one or more oxidative stress markers, an abnormally high concentration of reactive oxygen species, immune system overactivation, or any combination thereof in the subject before administration of the dual N-type and L-type calcium channel blocker selective for the N-type calcium channel.

Embodiment 52. The method of any one of embodiments 1-51, further comprising determining a reduction in oxidative stress, a reduction in upregulation of superoxide dismutase, a reduction in upregulation of one or more oxidative stress markers, a reduction in reactive oxygen species, a reduction in immune system activation, or any combination thereof in the subject after administration of the dual N-type and L-type calcium channel blocker selective for the N-type calcium channel.

Embodiment 53. The method of any one of embodiments 1-52, wherein the one or more oxidative stress markers are selected from nitrotyrosine, isoprostane, malondialdehyde, myeloperoxidase, oxidized low density lipoproteins, and S-glutathionylation of haemoglobin.

Embodiment 54. The method of any one of embodiments 1-53, further comprising determining that the subject has microvascular pathology and/or ischemia reperfusion injury before administration of the dual N-type and L-type calcium channel blocker selective for the N-type calcium channel.

Embodiment 55. The method of any one of embodiments 1-54, further comprising determining that the microvascular pathology and/or ischemia reperfusion injury in the subject is treated, improved, or ameliorated after administration of the dual N-type and L-type calcium channel blocker selective for the N-type calcium channel.

Embodiment 56. The method of any one of embodiments 1-55, further comprising determining endothelial dysfunction in the subject before administration of the dual N-type and L-type calcium channel blocker selective for the N-type calcium channel.

Embodiment 57. The method of any one of embodiments 1-56, wherein endothelial function in the subject is improved after administration of the dual N-type and L-type calcium channel blocker selective for the N-type calcium channel.

Embodiment 58. The method of any one of embodiments 1-57, further comprising determining an abnormally high stress response to cold sensitization and/or exposure in the subject before administration of the dual N-type and L-type calcium channel blocker selective for the N-type calcium channel.

Embodiment 59. The method of any one of embodiments 1-58, further comprising determining a reduction in stress response to cold sensitization and/or exposure in the subject after administration of the dual N-type and L-type calcium channel blocker selective for the N-type calcium channel.

Embodiment 60. The method of any one of embodiments 1-59, further comprising administering an additional therapeutic agent to the subject.

Embodiment 61. The method of embodiment 60, wherein the additional therapeutic agent is selected from the group consisting of: riociguat, amlodipine, pregabalin, gabapentin, nifedipine, nicardipine, conotoxins, cadmium, caroverine, levetiracetam, lamotrigine, NP078585, pregabalin, TROX-1, non-steroidal anti-inflammatory agents, valsartan, dimethylsulfoxide, N-acetylcysteine, N-acetyl cysteine, 4-hydroxy-2,2,6,6-tetramethylpiperidin-1-oxyl, magnesium sulfate, ziconotide, analgesics, anesthetics, anticonvulsants, antidepressants, oral muscle relaxants, corticosteroids, calcitonin, bisphosphonates, cyclooxygenase (COX)-2 inhibitors, free-radical scavenger agents, prednisolone, prednisone, oral steroids, opioids, riociguat, amlodipine, pregabalin, alendronate, pamidronate, gabapentin, nifedipine, nicardipine, bupivacaine, epinephrine, caroverine, ascorbic acid, valsartan, dimethylsulfoxide, N-acetylcysteine, phenoxybenzamine.

Embodiment 62. The method of any one of embodiments 1-61, wherein vasoconstriction in the subject is reduced after administering the dual N-type and L-type calcium channel blocker selective for the N-type calcium channel to the subject.

Embodiment 63. The method of any one of embodiments 1-62, wherein after administration of the dual N-type and L-type calcium channel blocker selective for the N-type calcium channel to the subject, sympathetic tone diminution, direct smooth muscle relaxation, or both occur in the subject.

Embodiment 64. The method of any one of embodiments 1-63, wherein after administration of the dual N-type and L-type calcium channel blocker selective for the N-type calcium channel to the subject, burning pain, body temperature changes of the subject, thermal hyperesthesia, thermal hyperalgesia, changes in skin or tissue color, edema, changes in skin turgor, ruble, pallor, cyanosis, vasospasm, or a combination thereof are treated in the subject.

Embodiment 65. The method of any one of embodiments 1-64, wherein after administration of the dual N-type and L-type calcium channel blocker selective for the N-type calcium channel to the subject, nitric oxide is increased in the subject.

Embodiment 66. The method of any one of embodiments 1-65, wherein after administration of the dual N-type and L-type calcium channel blocker selective for the N-type calcium channel to the subject, oxidative stress in the subject is decreased.

Embodiment 67. The method of any one of embodiments 1-66, wherein after administration of the dual N-type and L-type calcium channel blocker selective for the N-type calcium channel to the subject, norepinephrine is reduced in the subject.

Embodiment 68. The method of any one of embodiments 1-67, wherein the subject is identified as normotensive; and wherein after administration of the dual N-type and L-type calcium channel blocker selective for the N-type calcium channel to the subject, the heart rate and systolic blood pressure of the subject remains substantially the same.

Embodiment 69. The method of any one of embodiments 1-67, wherein the subject is also diagnosed with hypertension; and wherein after administration of the dual N-type and L-type calcium channel blocker selective for the N-type calcium channel to the subject, the systolic blood pressure of the subject is reduced.

Embodiment 70. The method of embodiment 69, wherein the systolic blood pressure of the subject is reduced by greater than 1 mm Hg.

Embodiment 71. The method of embodiment 69, wherein the systolic blood pressure of the subject is reduced by greater than 10 mm Hg.

Embodiment 72. The method of any one of embodiments 1-71, wherein after administration of the dual N-type and L-type calcium channel blocker selective for the N-type calcium channel, the bone density of the subject does not decrease.

Embodiment 73. The method of any one of embodiments 1-72, further comprising selecting a subject identified or diagnosed as having reduced bone density for the treatment.

Embodiment 74. The method of embodiment 73, wherein the subject identified or diagnosed as having reduced bone density has osteoporosis.

Embodiment 75. The method of any one of embodiments 1-74, further comprising selecting a subject identified or diagnosed as having reduced renal function for the treatment.

Embodiment 76. The method of embodiment 75, wherein the renal function of the subject is not reduced after treatment.

Embodiment 77. The method of embodiment 76, wherein the renal function of the subject is improved after treatment.

Embodiment 78. The method of embodiment 77, wherein improved renal function comprises a reduction in intrarenal arterial stiffness, improved blood flow to the kidneys, increased expression levels of podocyte proteins, or any combination thereof.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating complex regional pain syndrome in a subject in need thereof, comprising administering to the subject a therapeutically effective amount of a dual N-type and L-type calcium channel blocker selective for the N-type calcium channel.

2. A method of treating pain in a subject in need thereof, comprising administering to the subject a therapeutically effective amount of a dual N-type and L-type calcium channel blocker selective for the N-type calcium channel; wherein the subject has complex regional pain syndrome.

3. The method of any one of claims 1-2, wherein the complex regional pain syndrome is identified or diagnosed in a leg of the subject.

4. The method of any one of claims 1-2, wherein the complex regional pain syndrome is identified or diagnosed in an arm of the subject.

5. The method of any one of claims 1-2, wherein the complex regional pain syndrome is associated with an injury in the subject.

6. The method of claim 5, wherein the injury is a bone fracture or a crushing injury to a nerve.

7. The method of any one of claims 1-2, wherein the complex regional pain syndrome is associated with a stroke in the subject.

8. The method of any one of claims 1-2, wherein the complex regional pain syndrome is associated with a heart attack in the subject.

9. The method of any one of claims 1-8, wherein the complex regional pain syndrome is complex regional pain syndrome Type I.

10. The method of any one of claims 1-8, wherein the complex regional pain syndrome is complex regional pain syndrome Type II.

11. The method of any one of claims 1-10, wherein after administration of the therapeutically effective amount of the dual N-type and L-type calcium channel blocker selective for the N-type calcium channel, the severity, frequency, and/or duration of burning or throbbing pain, sensitivity to touch or cold, swelling, increased or decreased perspiration, changes in skin temperature, changes in skin texture, changes in skin color, changes in growth rate of hair and/or nails, joint stiffness, muscle spasm, muscle weakness, muscle atrophy, excess bone growth, impaired movement, decreased range of motion of a joint, or any combination thereof are reduced in the subject.

12. The method of claim 2, wherein the pain is neuropathic pain.

13. The method of any one of claims 1-12, wherein the dual N-type and L-type calcium channel blocker selective for the N-type calcium channel exhibits at least a 50-fold selectivity for the N-type calcium channel over an L-type calcium channel.

14. The method of any one of claims 1-13, wherein the dual N-type and L-type calcium channel blocker selective for the N-type calcium channel exhibits a 50-fold to 100-fold selectivity for the N-type calcium channel over an L-type calcium channel.

15. The method of any one of claims 1-14, wherein the dual N-type and L-type calcium channel blocker selective for the N-type calcium channel is selected from the group consisting of: cilnidipine, Z-160, CNV2197944, or pharmaceutically acceptable salts thereof.

16. The method of any one of claims 1-15, wherein the dual N-type and L-type calcium channel blocker selective for the N-type calcium channel is cilnidipine or a pharmaceutically acceptable salt thereof.

17. The method of claim 16, wherein the dosage of the cilnidipine is from about 5 mg to about 25 mg.

18. The method of claim 16, wherein the dosage of the cilnidipine is from about 9 mg to about 21 mg.

19. The method of claim 16, wherein the dosage of the cilnidipine is about 10 mg.

20. The method of claim 16, wherein the dosage of the cilnidipine is about 20 mg.

21. The method of any one of claims 1-20, wherein the dual N-type and L-type calcium channel blocker selective for the N-type calcium channel is administered orally.

22. The method of any one of claims 1-21, wherein each administration of the dual N-type and L-type calcium channel blocker selective for the N-type calcium channel is separated by at least about 8 hours.

23. The method of any one of claims 1-22, wherein each administration of the dual N-type and L-type calcium channel blocker selective for the N-type calcium channel is separated by at least about 24 hours.

24. The method of any one of claims 1-23, wherein each administration of the dual N-type and L-type calcium channel blocker selective for the N-type calcium channel is separated by at least about 48 hours.

25. The method of any one of claims 1-24, wherein each administration of the dual N-type and L-type calcium channel blocker selective for the N-type calcium channel is separated by at least about 1 week.

26. The method of any one of claims 1-25, wherein the period of administration of the dual N-type and L-type calcium channel blocker selective for the N-type calcium channel is from about 1 day to about 1 month.

27. The method of any one of claims 1-25, wherein the period of administration of the dual N-type and L-type calcium channel blocker selective for the N-type calcium channel is from about 1 day to about two weeks.

28. The method of any one of claims 1-25, wherein the period of administration of the dual N-type and L-type calcium channel blocker selective for the N-type calcium channel is about two weeks.

29. The method of any one of claims 1-25, wherein the period of administration of the dual N-type and L-type calcium channel blocker selective for the N-type calcium channel is about 12 days.

30. The method of any one of claims 1-25, wherein the period of administration of the dual N-type and L-type calcium channel blocker selective for the N-type calcium channel is about one week.

31. The method of any one of claims 1-30, wherein the dual N-type and L-type calcium channel blocker selective for the N-type calcium channel further inhibits a Nav 1.7 sodium channel.

32. The method of any one of claims 1-31, wherein the dual N-type and L-type calcium channel blocker selective for the N-type calcium channel further inhibits a TRPv-1 channel.

33. The method of any one of claims 1-32, further comprising determining that the subject has TRPv-1 overactivation.

34. The method of claim 33, wherein determining that the subject has TRPv-1 overactivation comprises determining an abnormally high TRP-v1 current density in the subject.

35. The method of any one of claims 1-34, wherein the subject has a clinical record showing TRPv-1 overactivation in the subject.

36. The method of any one of claims 1-35, further comprising determining a decrease in TRPv-1 activation or upregulation in the subject after administration of the dual N-type and L-type calcium channel blocker selective for the N-type calcium channel.

37. The method of any one of claims 1-36, further comprising determining an abnormally high inflammation in the subject before administration of the dual N-type and L-type calcium channel blocker selective for the N-type calcium channel.

38. The method of any one of claims 1-37, further comprising determining a reduction in inflammation in the subject after administration of the dual N-type and L-type calcium channel blocker selective for the N-type calcium channel.

39. The method of any one of claims 37-38, wherein the inflammation is generalized, neurogenic, or both.

40. The method of any one of claims 1-39, further comprising determining that the subject has abnormally elevated concentrations of TNF-α, IL-1B. IL2, IL-6, ET-I, cGRP, bradykinin, substance P, MMP9, CXCL13, or any combination thereof before administration of the dual N-type and L-type calcium channel blocker selective for the N-type calcium channel.

41. The method of any one of claims 1-40, further comprising determining a lower concentration of TNF-α, IL-1B. IL2, IL-6, ET-I, cGRP, bradykinin, substance P, MMP9, CXCL13, or any combination thereof in the subject after administration of the dual N-type and L-type calcium channel blocker selective for the N-type calcium channel.

42. The method of any one of claims 40-41, wherein the abnormally elevated concentrations of TNF-α, IL-1B. IL2, IL-6, ET-I, cGRP, bradykinin, substance P, MMP9, CXCL13, or any combination thereof are measured in the cerebrospinal fluid or the plasma of the subject.

43. The method of any one of claims 1-42, further comprising determining that the subject has an abnormally high immunologic response, an abnormally high number or concentration of non-specific autoantibodies to sympathetic receptors, or both before administration of the dual N-type and L-type calcium channel blocker selective for the N-type calcium channel.

44. The method of any one of claims 1-43, further comprising determining a reduction in immunologic response, a reduction in number or concentration of non-specific autoantibodies to sympathetic receptors, or both after administration of the dual N-type and L-type calcium channel blocker selective for the N-type calcium channel.

45. The method of any one of claims 1-44, further comprising determining autonomic dysregulation; an abnormally high number of sympathetic receptors; an abnormally high concentration of circulating catecholamines; sympathetic hyperactivity; or any combination thereof in the subject before administration of the dual N-type and L-type calcium channel blocker selective for the N-type calcium channel.

46. The method of any one of claims 1-45, further comprising determining an improvement in autonomic functioning; a reduction in sympathetic receptors; a reduction in the concentration of circulating catecholamines; a reduction in sympathetic activity; or any combination thereof in the subject after administration of the dual N-type and L-type calcium channel blocker selective for the N-type calcium channel.

47. The method of any one of claims 1-46, further comprising determining an abnormally high central sensitization and neuroplasticity with increased substance P, bradykinin, and/or glutamate; an overexpression of spinal NMDA receptors; gabaminergic overactivity; or any combination thereof in the subject before administration of the dual N-type and L-type calcium channel blocker selective for the N-type calcium channel.

48. The method of any one of claims 1-47, further comprising determining a reduction in central sensitization and neuroplasticity with increased substance P, bradykinin, and/or glutamate; an overexpression of spinal NMDA receptors; gabaminergic overactivity; or any combination thereof in the subject after administration of the dual N-type and L-type calcium channel blocker selective for the N-type calcium channel.

49. The method of any one of claims 1-48, further comprising determining peripheral oversensitization; an abnormally high concentration of spinal N-methyl-D-aspartate; abnormally high concentration of spinal glutamate; upregulation in TNF-α and/or TRP-V1 activation; or any combination thereof in the subject before administration of the dual N-type and L-type calcium channel blocker selective for the N-type calcium channel.

50. The method of any one of claims 1-49, further comprising determining a reduction in peripheral sensitization; a reduction in spinal N-methyl-D-aspartate; a reduction in spinal glutamate; a reduction in upregulation of TNF-α and/or TRP-V1 activation; or any combination thereof in the subject after administration of the dual N-type and L-type calcium channel blocker selective for the N-type calcium channel.

51. The method of any one of claims 1-50, further comprising determining abnormally high oxidative stress, upregulation of superoxide dismutase, upregulation of one or more oxidative stress markers, an abnormally high concentration of reactive oxygen species, immune system overactivation, or any combination thereof in the subject before administration of the dual N-type and L-type calcium channel blocker selective for the N-type calcium channel.

52. The method of any one of claims 1-51, further comprising determining a reduction in oxidative stress, a reduction in upregulation of superoxide dismutase, a reduction in upregulation of one or more oxidative stress markers, a reduction in reactive oxygen species, a reduction in immune system activation, or any combination thereof in the subject after administration of the dual N-type and L-type calcium channel blocker selective for the N-type calcium channel.

53. The method of any one of claims 1-52, wherein the one or more oxidative stress markers are selected from nitrotyrosine, isoprostane, malondialdehyde, myeloperoxidase, oxidized low density lipoproteins, and S-glutathionylation of haemoglobin.

54. The method of any one of claims 1-53, further comprising determining that the subject has microvascular pathology and/or ischemia reperfusion injury before administration of the dual N-type and L-type calcium channel blocker selective for the N-type calcium channel.

55. The method of any one of claims 1-54, further comprising determining that the microvascular pathology and/or ischemia reperfusion injury in the subject is treated, improved, or ameliorated after administration of the dual N-type and L-type calcium channel blocker selective for the N-type calcium channel.

56. The method of any one of claims 1-55, further comprising determining endothelial dysfunction in the subject before administration of the dual N-type and L-type calcium channel blocker selective for the N-type calcium channel.

57. The method of any one of claims 1-56, wherein endothelial function in the subject is improved after administration of the dual N-type and L-type calcium channel blocker selective for the N-type calcium channel.

58. The method of any one of claims 1-57, further comprising determining an abnormally high stress response to cold sensitization and/or exposure in the subject before administration of the dual N-type and L-type calcium channel blocker selective for the N-type calcium channel.

59. The method of any one of claims 1-58, further comprising determining a reduction in stress response to cold sensitization and/or exposure in the subject after administration of the dual N-type and L-type calcium channel blocker selective for the N-type calcium channel.

60. The method of any one of claims 1-59, further comprising administering an additional therapeutic agent to the subject.

61. The method of claim 60, wherein the additional therapeutic agent is selected from the group consisting of: riociguat, amlodipine, pregabalin, gabapentin, nifedipine, nicardipine, conotoxins, cadmium, caroverine, levetiracetam, lamotrigine, NP078585, pregabalin, TROX-1, non-steroidal anti-inflammatory agents, valsartan, dimethylsulfoxide, N-acetylcysteine, N-acetyl cysteine, 4-hydroxy-2,2,6,6-tetramethylpiperidin-1-oxyl, magnesium sulfate, ziconotide, analgesics, anesthetics, anticonvulsants, antidepressants, oral muscle relaxants, corticosteroids, calcitonin, bisphosphonates, cyclooxygenase (COX)-2 inhibitors, free-radical scavenger agents, prednisolone, prednisone, oral steroids, opioids, riociguat, amlodipine, pregabalin, alendronate, pamidronate, gabapentin, nifedipine, nicardipine, bupivacaine, epinephrine, caroverine, ascorbic acid, valsartan, dimethylsulfoxide, N-acetylcysteine, phenoxybenzamine.

62. The method of any one of claims 1-61, wherein vasoconstriction in the subject is reduced after administering the dual N-type and L-type calcium channel blocker selective for the N-type calcium channel to the subject.

63. The method of any one of claims 1-62, wherein after administration of the dual N-type and L-type calcium channel blocker selective for the N-type calcium channel to the subject, sympathetic tone diminution, direct smooth muscle relaxation, or both occur in the subject.

64. The method of any one of claims 1-63, wherein after administration of the dual N-type and L-type calcium channel blocker selective for the N-type calcium channel to the subject, burning pain, body temperature changes of the subject, thermal hyperesthesia, thermal hyperalgesia, changes in skin or tissue color, edema, changes in skin turgor, ruble, pallor, cyanosis, vasospasm, or a combination thereof are treated in the subject.

65. The method of any one of claims 1-64, wherein after administration of the dual N-type and L-type calcium channel blocker selective for the N-type calcium channel to the subject, nitric oxide is increased in the subject.

66. The method of any one of claims 1-65, wherein after administration of the dual N-type and L-type calcium channel blocker selective for the N-type calcium channel to the subject, oxidative stress in the subject is decreased.

67. The method of any one of claims 1-66, wherein after administration of the dual N-type and L-type calcium channel blocker selective for the N-type calcium channel to the subject, norepinephrine is reduced in the subject.

68. The method of any one of claims 1-67, wherein the subject is identified as normotensive; and wherein after administration of the dual N-type and L-type calcium channel blocker selective for the N-type calcium channel to the subject, the heart rate and systolic blood pressure of the subject remains substantially the same.

69. The method of any one of claims 1-67, wherein the subject is also diagnosed with hypertension; and wherein after administration of the dual N-type and L-type calcium channel blocker selective for the N-type calcium channel to the subject, the systolic blood pressure of the subject is reduced.

70. The method of claim 69, wherein the systolic blood pressure of the subject is reduced by greater than 1 mm Hg.

71. The method of claim 69, wherein the systolic blood pressure of the subject is reduced by greater than 10 mm Hg.

72. The method of any one of claims 1-71, wherein after administration of the dual N-type and L-type calcium channel blocker selective for the N-type calcium channel, the bone density of the subject does not decrease.

73. The method of any one of claims 1-72, further comprising selecting a subject identified or diagnosed as having reduced bone density for the treatment.

74. The method of claim 73, wherein the subject identified or diagnosed as having reduced bone density has osteoporosis.

75. The method of any one of claims 1-74, further comprising selecting a subject identified or diagnosed as having reduced renal function for the treatment.

76. The method of claim 75, wherein the renal function of the subject is not reduced after treatment.

77. The method of claim 76, wherein the renal function of the subject is improved after treatment.

78. The method of claim 77, wherein improved renal function comprises a reduction in intrarenal arterial stiffness, improved blood flow to the kidneys, increased expression levels of podocyte proteins, or any combination thereof.
